(19) United States
(12) Patent Application Publication
(10) Pub. No.: US 2026/0337128 A1
(43) Pub. Date: Oct. 29, 2026

(54) HETEROGENOUS CATALYST COMPOSITIONS FOR ETHYLENE OLIGOMERIZATION

(71) Applicant: Chevron Phillips Chemical Company LP, The Woodlands, TX (US)

(72) Inventors: Michael S. Webster-Gardiner, Kingwood, TX (US); Steven M. Bischof, Kingwood, TX (US); Thomas B. Gunnoe, Charlotteville, VA (US); Fanji Kong, Charlotteville, VA (US)

(21) Appl. No.: 19/651,938

(22) Filed: Apr. 20, 2026

Related U.S. Application Data
(60) Provisional application No. 63/792,650, filed on Apr. 22, 2025.

(51) Int. Cl. C07C 2/34; B01J 21/04; B01J 21/08; B01J 21/18; B01J 29/06; B01J 31/14; B01J 31/18; B01J 31/24; B01J 35/30; B01J 35/40; B01J 35/61; B01J 35/63; B01J 37/02; B01J 37/04

(52) U.S. Cl. CPC C07C 2/34; B01J 21/04; B01J 21/08; B01J 21/18; B01J 29/06; B01J 31/143; B01J 31/1805; B01J 31/189; B01J 31/2404; B01J 35/394; B01J 35/40; B01J 35/612; B01J 35/613; B01J 35/615; B01J 35/617; B01J 35/618; B01J 35/633; B01J 35/635; B01J 35/638; B01J 37/0221; B01J 37/0236; B01J 37/04; B01J 2231/20; B01J 2531/31; B01J 2531/62; B01J 2531/842; B01J 2531/845; C07C 2531/22

(57) ABSTRACT

Heterogenous catalyst compositions for ethylene oligomerization processes are disclosed. These catalyst compositions comprise heteroatomic ligand transition metal complexes impregnated onto solid supports and maintain the same catalytic activity as that of analogous metal complexes within homogenous catalyst compositions. Oligomerization processes using the heterogenous catalyst compositions demonstrate advantages in downstream separations of the oligomer product from unreacted ethylene and the solid catalyst composition, as well as eliminating the need for catalyst kill agents associated with oligomerization processes that utilize homogenous catalyst systems.

### REFERENCE TO RELATED APPLICATION

This application claims the benefit of U.S. Provisional Patent Application No. 63/792,650, filed on Apr. 22, 2025, the disclosure of which is incorporated herein by reference in its entirety.

### TECHNICAL FIELD

The present disclosure relates generally to processes for preparing heterogenous catalyst compositions, and more particularly, to the use of the heterogenous catalyst compositions in ethylene oligomerization processes.

### BACKGROUND

Alpha olefins can be produced using an ethylene reactant and various combinations of catalyst systems and oligomerization processes. It can be beneficial for the catalyst system to be heterogenous, supported in the solid phase within the oligomerization reactor, to improve the downstream separation of products and excess reactants. Accordingly, it is to these ends that the present disclosure is generally directed.

### SUMMARY

This summary is provided to introduce a selection of concepts in a simplified form that are further described herein. This summary is not intended to identify required or essential features of the claimed subject matter. Nor is this summary intended to be used to limit the scope of the claimed subject matter.

Disclosed herein are catalyst compositions comprising a heteroatomic ligand transition metal complex, an activator, and a solid support. In certain aspects, the heteroatomic ligand transition metal complex can have any of the following Formulas (I)-(III):

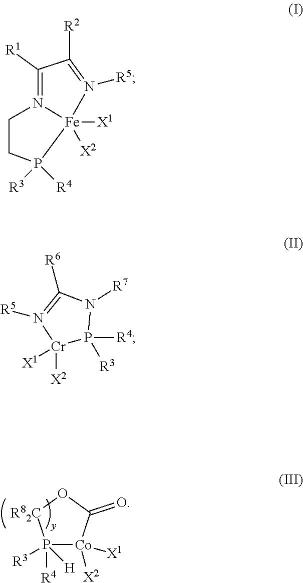

In each of Formulas (I)-(III), X¹ and X² independently can be a monoanionic ligand (e.g., a halogen, a carboxylate, a β-diketonate, a hydrocarboxide, a nitrate, or a chlorate). R¹ and R², where present, independently can be H or a substituted or unsubstituted C₁ to C₁₈ hydrocarbyl group. R³ and R⁴ independently can be a substituted or unsubstituted C₁ to C₂₀ organyl group. Where present, R⁵, R⁶, and R⁷ independently can be a C₁ to C₂₀ organyl group. In Formula (III), y can be 0, 1, or 2, and each R⁸, when present, independently is H or a substituted or unsubstituted C₁ to C₁₈ hydrocarbyl group.

Processes for preparing heterogeneous catalyst compositions are also disclosed herein and can comprise (a) contacting any heteroatomic ligand transition metal complex disclosed herein with an activator, typically in a hydrocarbon solvent, to form an activated catalyst, (b) contacting the activated catalyst and a solid support to form a supported catalyst, and (c) drying the supported catalyst to form the heterogenous catalyst composition.

Heterogenous catalyst compositions prepared as disclosed herein may be employed within oligomerization processes. These oligomerization processes can comprise (I) contacting ethylene, a heterogenous catalyst composition, and optionally hydrogen, in an oligomerization reactor, (II) forming an oligomer product in the oligomerization reactor, the oligomer product comprising C₄ to C₂₀ ethylene oligomers, and (III) discharging an effluent stream from the oligomerization reactor, the effluent stream comprising unreacted ethylene and the oligomer product. Oligomerization processes utilizing heterogenous catalyst compositions can provide practical benefits to the handling of the catalyst composition within the reactor system during oligomerization, for instance by retaining the catalyst within a reaction zone of an oligomerization reactor. Heterogenous catalyst compositions also can improve downstream treatment of the effluent stream by allowing an easier separation and isolation of the oligomer product from the catalyst composition, as well as a streamlined recovery and reuse of the unreacted ethylene and the heterogenous catalyst composition itself. Further, and unexpectedly, the disclosed heterogenous catalyst compositions and oligomerization processes have comparable activity and selectivity to that of otherwise identical unsupported homogenous catalyst systems and oligomerization processes.

Both the foregoing summary and the following detailed description provide examples and are explanatory only. Accordingly, the foregoing summary and the following detailed description should not be considered to be restrictive. Further, features or variations may be provided in addition to those set forth herein. For example, certain aspects may be directed to various feature combinations and sub-combinations described in the detailed description.

### BRIEF DESCRIPTION OF THE FIGURES

The following figures form part of the present specification and is included to further demonstrate certain aspects of the present disclosure. The disclosure may be better understood by reference to these figures in combination with the detailed description.

FIG. 1A presents a chart showing the comparison of results using different Fe catalysts (Examples 11-13), with respect to the amount of oligomers produced.

FIG. 1B presents a chart showing the selectivity of oligomers produced using different Fe catalysts (Examples 11-13).

FIG. 2A presents a chart showing the results of Example 14, with respect to the amount of oligomers produced in seven consecutive ethylene oligomerization reactions.

FIG. 2B presents a chart showing the results of Example 14, with respect to the selectivity of oligomers produced.

FIG. 2C presents a chart showing the reactor temperature for the three separate trials using Example 14.

FIG. 3A presents a chart showing the results of a first trial of Example 15, with respect to the amount of oligomers produced, in four consecutive ethylene oligomerization reactions with house nitrogen purge the catalyst chamber between runs.

FIG. 3B presents a chart showing the results of the first trial of Example 15, with respect to the selectivity of oligomers produced, in four consecutive ethylene oligomerization reactions with house nitrogen purge the catalyst chamber between runs.

FIG. 3C presents a chart showing the results of a second trial of Example 15, with respect to the amount of oligomers produced, in four consecutive ethylene oligomerization reactions with house nitrogen purge the catalyst chamber between runs.

FIG. 3D presents a chart showing the results of the second trial of Example 15, with respect to the selectivity of oligomers produced, in four consecutive ethylene oligomerization reactions with house nitrogen purge the catalyst chamber between runs.

FIG. 4A presents a chart showing the results of Example 16, with respect to the amount of oligomers produced in four consecutive ethylene oligomerization reactions with cylinder nitrogen purge the catalyst chamber between runs.

FIG. 4B presents a chart showing the results of Example 16, with respect to the selectivity of oligomers produced, in four consecutive ethylene oligomerization reactions with cylinder nitrogen purge the catalyst chamber between runs.

FIG. 5A presents a chart showing the results of Example 17, with respect to the amount of oligomers produced using a 1 hour reaction time.

FIG. 5B presents a chart showing the results of Example 17, with respect to the selectivity of oligomers produced using a 1 hour reaction time.

FIG. 5C presents a chart showing the results of Example 17 with respect to the amount of oligomers produced using a 20-minute reaction time.

FIG. 5D presents a chart showing the results of Example 17, with respect to the selectivity of oligomers produced using a 20-minute reaction time.

FIG. 6A presents a chart showing the comparison of results of batch versus flow reactions Example 18, with respect to the amount of oligomers produced using a 20 minute reaction time.

FIG. 6B presents a chart showing the comparison of results of batch versus flow reactions Example 18, with respect to the selectivity of oligomers produced using a 20 minute reaction time.

FIG. 7A presents charts showing the results of Examples 19-20 with respect to the amount and selectivity of oligomers produced using a 20 minute reaction time and a 60 minute reaction time, for each of a first run using the catalyst, and a second run reusing the catalyst.

FIG. 7B presents charts showing the results of Examples 21-22 with respect to the amount and selectivity of oligomers produced using various flow rates, for each of a first run using the catalyst, and a second run reusing the catalyst.

FIG. 8A presents a chart showing the results of Example 23-24 comparing different flowrates, with respect to the turnover frequency of oligomers produced quantified in gas phase.

FIG. 8B presents a chart showing the results of Example 23-24, with respect to the selectivity of oligomers produced.

FIG. 9A presents a chart showing the results of Example 25-27 comparing different solid supports using flow reactor monitoring the products in gas-phase, with respect to the turnover frequency of oligomers produced.

FIG. 9B presents a chart showing the results of Example 25-27 comparing different solid supports using flow reactor monitoring the products in gas-phase, with respect to the selectivity of oligomers produced.

FIG. 10A presents a chart showing the results of Example 28, demonstrating the longevity, reusability, and reactivation using MMAO-3A of the activated carbon supported Fe-4 catalyst using flow reactor monitoring the products in gas-phase, with respect to the turnover frequency.

FIG. 10B presents a chart showing the results of Example 28 with respect to the reactor temperature.

FIG. 10C presents a chart showing the results of Example 28 with respect to the selectivity of α-C₄ oligomers produced.

FIG. 10D presents a chart showing the results of Example 28 with respect to the selectivity of α-C₆ oligomers produced.

FIG. 11A presents a chart showing the results of Example 29, demonstrating the reusability of the bulk synthesized activated carbon supported Fe-4 catalyst using flow reactor monitoring the products in gas-phase, with respect to (top left) the turnover frequency of oligomers produced.

FIG. 11B presents a chart showing the results of Example 28 with respect to the reactor temperature in each of three trials.

FIG. 11C presents a chart showing the results of Example 28 with respect to the selectivity of α-C₄ oligomers produced.

FIG. 11D presents a chart showing the results of Example 28 with respect to the selectivity of α-C₆ oligomers produced.

While the aspects disclosed herein are susceptible to various modifications and alternative forms, only a few specific aspects have been shown by way of example in the drawings and described in detail below. The figures and detailed description of specific aspects are not intended to limit the breadth or scope of the inventive concepts or the appended claims in any manner. Rather, the figures and detailed description are provided to illustrate the inventive concepts to a person of ordinary skill in the art and to enable such person to make and use the inventive concepts.

### DEFINITIONS

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology, 2nd Ed (1997), can be applied, as long as that definition does not conflict with any other disclosure or definition applied herein, or render indefinite or non-enabled any claim to which that definition is applied. To the extent that any definition or usage provided by any document incorporated herein by reference conflicts with the definition or usage provided herein, the definition or usage provided herein controls.

Herein, features of the subject matter are described such that, within particular aspects, a combination of different features can be envisioned. For each and every aspect and each and every feature disclosed herein, all combinations that do not detrimentally affect the compounds, compositions, processes, or methods described herein are contemplated with or without explicit description of the particular combination. Additionally, unless explicitly recited otherwise, any aspect or feature disclosed herein can be combined to describe inventive compounds, compositions, processes, or methods consistent with the present disclosure.

In this disclosure, while compositions and processes/methods are described in terms of “comprising” various materials or components and steps, the compositions and processes/methods also can “consist essentially of” or “consist of” the various materials or components and steps, unless stated otherwise. For example, a catalyst composition consistent with aspects of the present disclosure can comprise; alternatively, can consist essentially of, or alternatively, can consist of; a heteroatomic ligand transition metal complex, an activator, and a solid support. The terms “a,” “an,” and “the” are intended to include plural alternatives, e.g., at least one, unless otherwise specified. For instance, the disclosure of “an aluminoxane compound” is meant to encompass one, or mixtures or combinations of two or more, aluminoxane compound(s), unless otherwise specified.

For any generic or specific compound or group disclosed herein, any name or structure presented is intended to encompass all conformational isomers, regioisomers, stereoisomers, and mixtures thereof that can arise from a particular set of substituents, unless otherwise specified. The name or structure also encompasses all enantiomers, diastereomers, and other optical isomers (if there are any), whether in enantiomeric or racemic forms, as well as mixtures of stereoisomers, as would be recognized by a skilled artisan, unless otherwise specified. For example, a general reference to hexene (or hexenes) includes all linear or branched, acyclic or cyclic, hydrocarbon compounds having six carbon atoms and 1 carbon-carbon double bond; a general reference to pentane includes n-pentane, 2-methyl-butane, and 2,2-dimethylpropane; and a general reference to a butyl group includes an n-butyl group, a sec-butyl group, an iso-butyl group, and a t-butyl group.

The terms “contacting” and “combining” are used herein to describe compositions and processes/methods in which the materials are contacted or combined together in any order, in any manner, and for any length of time, unless otherwise specified. For example, the materials can be blended, mixed, slurried, dissolved, reacted, treated, impregnated, compounded, or otherwise contacted or combined in some other manner or by any suitable method or technique.

In the processes disclosed and claimed herein, and not limited thereto, a product in one step can be used as a reactant in another step, or a material can be separated from a reaction mixture. For example, when unreacted ethylene is removed (or separated, or isolated) from an effluent stream, “all or any portion of” the unreacted ethylene can be removed (or separated, or isolated) from the effluent stream, even if not specifically stated so. Likewise, all process steps in the specification and in the claims should be interpreted in a manner similar to this example.

The term “hydrocarbon” whenever used in this specification and claims refers to a compound containing only carbon and hydrogen, whether saturated or unsaturated. Other identifiers can be utilized to indicate the presence of particular groups in the hydrocarbon (e.g., halogenated hydrocarbon indicates that the presence of one or more halogen atoms replacing an equivalent number of hydrogen atoms in the hydrocarbon). The term “hydrocarbyl group” is used herein in accordance with the definition specified by IUPAC: a univalent group formed by removing a hydrogen atom from a hydrocarbon (that is, a group containing only carbon and hydrogen). Non-limiting examples of hydrocarbyl groups include alkyl, alkenyl, aryl, and aralkyl groups, amongst other groups.

The term “organyl group” is used herein in accordance with the definition specified by IUPAC: an organic substituent group, regardless of functional type, having one free valence at a carbon atom. An “organic group” refers to a generalized group formed by removing one or more hydrogen atoms from carbon atoms of an organic compound. Thus, an “organyl group” and an “organic group” can contain organic functional group(s) and/or atom(s) other than carbon and hydrogen, that is, an organic group can comprise functional groups and/or atoms in addition to carbon and hydrogen. An “organyl group,” or “organic group” can be aliphatic, (inclusive of being cyclic or acyclic, or linear or branched), or can be aromatic.

The term “olefin” includes aliphatic and aromatic, cyclic and acyclic, and/or linear and branched compounds having at least one carbon-carbon double bond that is not part of an aromatic ring or ring system, unless specifically stated otherwise. Olefins having only one, only two, only three, etc., carbon-carbon double bonds can be identified by use of the term “mono,” “di,” “tri,” etc., within the name of the olefin. Olefins can be further identified by the position of the carbon-carbon double bond(s). The term “alpha olefin” as used herein refers to any olefin that has a double bond between the first and second carbon atom of a contiguous chain of carbon atoms. The term “alpha olefin” includes linear and branched alpha olefins and alpha olefins which have more than one non-aromatic carbon-carbon double bond, unless expressly stated otherwise. The term “normal alpha olefin” as used herein refers to a linear aliphatic hydrocarbon mono-olefin having a double bond between the first and second carbon atom.

The term “oligomer” refers to a compound that contains from 2 to 20 monomer units. The terms “oligomerization product” and “oligomer product” include all products made by the “oligomerization” process, including the “oligomers” and products which are not “oligomers” (e.g., products which contain more than 20 monomer units, or solid polymer), but exclude other non-oligomer components of an oligomerization reactor effluent stream, such as unreacted ethylene and hydrogen, amongst other components.

The terms “catalyst composition,” “catalyst mixture,” “catalyst system,” and the like, do not depend upon the actual product or composition resulting from the contact or reaction of the initial components of the disclosed or claimed catalyst composition (or catalyst mixture or catalyst system), the nature of the activated catalyst composition, or the fate of the metal complex, activator, and the solid support after combining these components. Therefore, the terms “catalyst composition,” “catalyst mixture,” “catalyst system,” and the like, encompass the initial starting components of the composition, as well as whatever product(s) may result from contacting these initial starting components, and this is inclusive of both heterogenous and homogenous catalyst systems or compositions. The terms “catalyst composition,” “catalyst mixture,” “catalyst system,” and the like, may be used interchangeably throughout this disclosure.

Several types of ranges are disclosed in the present disclosure. When a range of any type is disclosed or claimed, the intent is to disclose or claim individually each possible number that such a range could reasonably encompass, including end points of the range as well as any sub-ranges and combinations of sub-ranges encompassed therein. For example, the molar ratio of Al:transition metal in the activated catalyst composition can be in various ranges. By a disclosure that the molar ratio can be in a range from 10:1 to 5,000:1, the intent is to recite that the molar ratio can be any molar ratio within the range and, for example, can include any range or combination of ranges from 10:1 to 5,000:1, such as from 50:1 to 3,000:1, from 75:1 to 2,000:1, from 100:1 to 2,000:1, or from 100:1 to 1,000:1, and so forth. Likewise, all other ranges disclosed herein should be interpreted in a manner similar to this example.

In general, an amount, size, formulation, parameter, range, or other quantity or characteristic is “about” or “approximate” whether or not expressly stated to be such. Whether or not modified by the term “about” or “approximately,” the claims include equivalents to the quantities or characteristics.

Although any methods and materials similar or equivalent to those described herein can be used in the practice or testing of the disclosure, the typical methods and materials are herein described.

All publications and patents mentioned herein are incorporated herein by reference in their entirety for the purpose of describing and disclosing, for example, the constructs and methodologies that are described in the publications and patents, which might be used in connection with the present disclosure.

### DETAILED DESCRIPTION

Disclosed herein are heterogenous catalyst compositions containing heteroatomic ligand transition metal complexes and ethylene oligomerization processes utilizing the heterogenous catalyst compositions to produce C₄+ oligomers.

### Heterogenous Catalyst Compositions

Heterogenous catalyst compositions are disclosed herein for the oligomerization of ethylene. Generally, these catalyst compositions can comprise a heteroatomic ligand transition metal complex as a catalyst or precatalyst (e.g., Formulas (I)-(III) described independently below), an activator, and a solid support.

Suitable heteroatomic ligand transition metal complexes can include, without limitation, an iron complex with a α-diimine iron salt complex (e.g., Formula (I)), a chromium complex with an N²-phosphinyl formamidine ligand, an N²-phosphinyl amidine ligand, an N²-phosphinyl guanidine ligand, or a heterocyclic 2-[(phosphinyl)aminyl]imine ligand (e.g., Formula (II)), and a phosphine-Co complex (e.g., Formula (III)), as described in further detail below.

In certain aspects, the catalyst complex can comprise an α-diimine iron salt complex as shown in Formula (I):

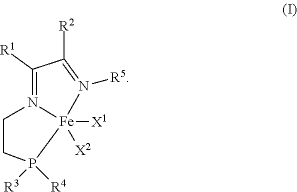

In Formula (I), X¹ and X² independently can be a monoanionic ligand, R¹ and R² independently can be H or a substituted or unsubstituted C₁ to C₁₈ hydrocarbyl group, and R³ and R⁴ independently can be a substituted or unsubstituted C₁ to C₂₀ organyl group. In certain aspects, X¹ and X² can be Cl. Additionally or alternatively, R¹, R², R³, R⁴, and R⁵ independently can be a substituted or unsubstituted C₆ to C₁₂ aryl group. Optionally, R¹ and R² can be joined to form a ring or ring system. Additional α-diimine iron salt complexes encompassed by Formula (I) and/or iron complexes that can be utilized in the catalyst compositions contemplated herein can be found in, but not limited to, U.S. Pat. Nos. 7,268,096 and 10,604,457, and U.S. Patent Publication No. 2024/0360052.

Alternatively, in other aspects, the catalyst complex can comprise a chromium-based complex having Formula (II):

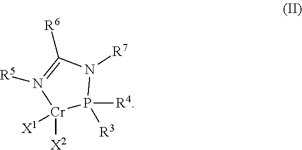

R³ and R⁴ in Formula (II) can be as described above for Formula (I), and R⁵, R⁶, and R⁷ likewise independently can be a C₁ to C₂₀ organyl group, such as a substituted or unsubstituted C₆ to C₁₂ aryl group. Similarly, X¹ and X² in Formula (II) independently can be a monoanionic ligand (e.g., a halogen, a carboxylate, a β-diketonate, a hydrocarboxide, a nitrate, or a chlorate), and in certain aspects, both X¹ and X² can be Cl. In a particular aspect, R⁶ can be NR^{6a}R^{6b} where R^{6a} and R^{6b} independently can be a C₁ to C₂₀ organyl group, each optionally forming a linking group with R⁵, R⁷, or each other. Additional N²-phosphinylguanidine chromium complexes and N²-phosphinylformadine chromium complexes that are encompassed by Formula (II) and/or can be utilized in the catalyst composition contemplated herein can be found in, but not limited to, U.S. Pat. Nos. 8,865,610 and 9,962,689.

In yet another aspect, the catalyst complex can comprise a cobalt-based complex having Formula (III):

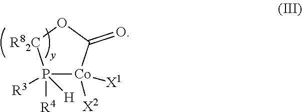

In Formula (III), X¹ and X² independently can be a monoanionic ligand as described for Formula (I) above (e.g., X¹ and X² can be Cl). R³ and R⁴ independently can be an organyl group as described for Formula (I) above (e.g., a substituted or unsubstituted C₆ to C₁₂ aryl group). The integer y can be 0, 1, or 2, and each R⁸, when present, independently can be H or a substituted or unsubstituted C₁ to C₁₈ hydrocarbyl group. Additional transition metal complexes that are encompassed by Formula (III) and/or can be utilized in the catalyst compositions contemplated herein can be found in, but not limited to, U.S. Pat. No. 10,519,077 and references cited therein.

Referring to each of Formulas (I)-(III) above, X¹ and X² independently can be a halogen (e.g., fluorine or chlorine), a carboxylate, a β-diketonate, a hydrocarboxide, a nitrate, or a chlorate. The hydrocarboxide can be an alkoxide, an aryloxide, or an aralkoxide. Generally, any carboxylate of the catalyst complex independently can be a C₂ to C₂₀ carboxylate, or alternatively, a C₂ to C₁₀ carboxylate. In an aspect, each carboxylate independently can be acetate, a propionate, a butyrate, a pentanoate, a hexanoate, a heptanoate, an octanoate, a nonanoate, a decanoate, an undecanoate, or a dodecanoate; or alternatively, a pentanoate, a hexanoate, a heptanoate, an octanoate, a nonanoate, a decanoate, an undecanoate, or a dodecanoate. In some aspects, each carboxylate independently can be acetate, propionate, n-butyrate, valerate (n-pentanoate), neo-pentanoate, capronate (n-hexanoate), n-heptanoate, caprylate (n-octanoate), 2-ethylhexanoate, n-nonanoate, caprate (n-decanoate), n-undecanoate, or laurate (n-dodecanoate); alternatively, valerate (n-pentanoate), neo-pentanoate, capronate (n-hexanoate), n-heptanoate, caprylate (n-octanoate), 2-ethylhexanoate, n-nonanoate, caprate (n-decanoate), n-undecanoate, or laurate (n-dodecanoate); alternatively, capronate (n-hexanoate); alternatively, n-heptanoate; alternatively, caprylate (n-octanoate); or alternatively, 2-ethylhexanoate. In some aspects, the carboxylate can be trifluoroacetate. In other aspects, the carboxylate can be triflate. In other aspects, two or more of X¹ and X² can be joined to form a dianionic or trianionic or polyanionic ligand to balance the oxidation state of the respective transition metal. When present, each β-diketonate of the catalyst complex independently can be any C₅ to C₂₀ a β-diketonate; or alternatively, any C₅ to C₁₀ β-diketonate. In an aspect, each β-diketonate independently can be acetylacetonate (i.e., 2,4-pentanedionate), hexafluoroacetylacetonate (i.e., 1,1,1,5,5,5-hexafluoro-2,4-pentanedionate), or benzoylacetonate; alternatively, acetylacetonate; alternatively, hexafluoroacetylacetonate; or alternatively, benzoylacetonate.

Generally, each hydrocarboxide of the catalyst complex independently can be any C₁ to C₂₀ hydrocarboxide; or alternatively, any C₁ to C₁₀ hydrocarboxide. In an aspect, each hydrocarboxide independently can be a C₁ to C₂₀ alkoxide; alternatively, a C₁ to C₁₀ alkoxide; alternatively, a C₆ to C₂₀ aryloxide; or alternatively, a C₆ to C₁₀ aryloxide. In an aspect, each alkoxide independently can be methoxide, ethoxide, a propoxide, or a butoxide; alternatively, methoxide, ethoxide, isopropoxide, or tert-butoxide; alternatively, methoxide; alternatively, ethoxide; alternatively, an iso-propoxide; or alternatively, a tert-butoxide. In an aspect, the aryloxide can be phenoxide.

In accordance with further descriptions of compounds within the references cited above, R¹, R², R³, R⁴, R⁵, R⁶, R⁷, and R⁸ are independently described herein and can be utilized in any combination and without limitation to further describe the catalyst complexes of Formulas (I)-(III) above.

Generally, R³, R⁴, R⁵, R⁶, and R⁷ independently can be an organyl group; alternatively, an organyl group consisting of inert functional groups; or alternatively, a hydrocarbyl group. In an aspect, the organyl group can be a C₁ to C₂₀, a C₁ to C₁₅, a C₁ to C₁₀, or a C₁ to C₅ organyl group. In an aspect, the organyl group consisting of inert functional groups can be a C₁ to C₂₀, a C₁ to C₁₅, a C₁ to C₁₀, or a C₁ to C₅ organyl group consisting of inert functional groups. In an aspect, R¹, R², R³, R⁴, R⁵, R⁶, R⁷, and R⁸ independently can be a C₁ to C₁₈, a C₁ to C₁₅, a C₁ to C₁₀, or a C₁ to C₅ hydrocarbyl group. In other aspects, R¹, R², R³, R⁴, R⁵, R⁶, R⁷, and R⁸ independently can be an alkyl group, a substituted alkyl group, a cycloalkyl group, a substituted cycloalkyl group, an aryl group, a substituted aryl group, an aralkyl group, or a substituted aralkyl group; alternatively, an alkyl group or a substituted alkyl group; alternatively, a cycloalkyl group or a substituted cycloalkyl group; alternatively, an aryl group or a substituted aryl group; alternatively, an aralkyl group or a substituted aralkyl group; alternatively, an alkyl group, a cycloalkyl group, an aryl group, or an aralkyl group; alternatively, an alkyl group; alternatively, a substituted alkyl group, alternatively, a cycloalkyl group; alternatively, a substituted cycloalkyl group; alternatively, an aryl group; alternatively, a substituted aryl group; alternatively, an aralkyl group; or alternatively, a substituted aralkyl group.

Generally, R³ and R⁴ independently can be an organyl group; alternatively, an organyl group consisting of inert functional groups; or alternatively, a hydrocarbyl group. In an aspect, the R³ and/or R⁴ organyl groups can be a C₁ to C₂₀, a C₁ to C₁₅, a C₁ to C₁₀, or a C₁ to C₅ organyl group. In an aspect, the R³ and/or R⁴ organyl groups consisting of inert functional groups can be a C₁ to C₂₀, a C₁ to C₁₅, a C₁ to C₁₀, or a C₁ to C₅ organyl group consisting of inert functional groups. In an aspect, the R³ and/or R⁴ hydrocarbyl groups can be a, a C₁ to C₁₈, a C₁ to C₁₅, a C₁ to C₁₀, or a C₁ to C₅ hydrocarbyl group. In an aspect, R³ and R⁴ independently can be an alkyl group, a substituted alkyl group, a cycloalkyl group, a substituted cycloalkyl group, an aryl group, a substituted aryl group, an aralkyl group, or a substituted aralkyl group; alternatively, an alkyl group or a substituted alkyl group; alternatively, a cycloalkyl group or a substituted cycloalkyl group; alternatively, an aryl group or a substituted aryl group; alternatively, an aralkyl group or a substituted aralkyl group; alternatively, an alkyl group, a cycloalkyl group, an aryl group, or an aralkyl group; alternatively, an alkyl group; alternatively, a substituted alkyl group, alternatively, a cycloalkyl group; alternatively, a substituted cycloalkyl group; alternatively, an aryl group; alternatively, a substituted aryl group; alternatively, an aralkyl group; or alternatively, a substituted aralkyl group.

Specific organyl and hydrocarbyl selections for each of R¹, R², R³, R⁴, R⁵, R⁶, R⁷, and R⁸ in the complexes of Formulas (I)-(III) can include those recited in U.S. Pat. Nos. 10,604,457, 10,519,077, and 11,230,514, though are not included in the interest of brevity.

Also contemplated herein are cobalt complexes with NNN pincer-type ligands. Although these complexes do not necessarily fit within the Formulas (I)-(III) above, their structures are generally known in the art and can include those shown below, as well as their variants and derivatives.

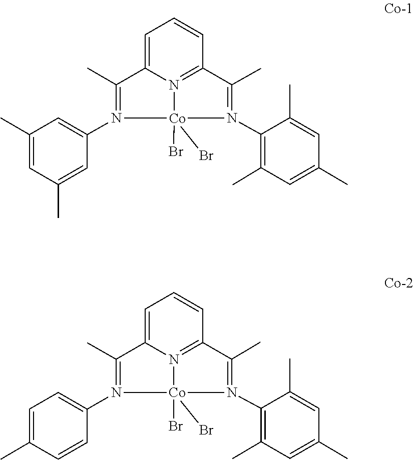

In a non-limiting aspect, catalyst compositions disclosed herein can comprise any activator suitable to generate an active form of the catalyst compound present as a precatalyst during preparation of the catalyst composition. In an aspect, the activator utilized in the catalyst systems can comprise, can consist essentially of, or can consist of, any aluminoxane which in conjunction with the heteroatomic ligand chromium complex can catalyze the formation of an oligomer product from ethylene. In a non-limiting aspect, the aluminoxane can have a repeating unit characterized by Formula (IV):

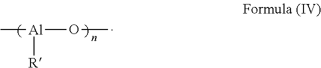

In Formula (IV), R′ is a linear or branched alkyl group. Alkyl groups of aluminoxanes (and alkylaluminum compounds) are independently described herein and can be utilized without limitation to further describe the aluminoxanes having Formula (IV) and/or the alkylaluminum compounds. Generally, n of Formula (IV) can be greater than 1; or alternatively, greater than 2. In an aspect, n can range from 2 to 15; or alternatively, range from 3 to 10.

In a non-limiting aspect, the aluminoxane can comprise, consist essentially of, or consist of, methylaluminoxane (MAO), ethylaluminoxane, modified methylaluminoxane (MMAO), n-propylaluminoxane, iso-propyl-aluminoxane, n-butylaluminoxane, sec-butylaluminoxane, iso-butylaluminoxane, t-butylaluminoxane, 1-pentylaluminoxane, 2-pentylaluminoxane, 3-pentyl-aluminoxane, iso-pentyl-aluminoxane, neopentylaluminoxane, or mixtures thereof. In some non-limiting aspects, the aluminoxane can comprise, consist essentially of, or consist of, methylaluminoxane (MAO), modified methylaluminoxane (MMAO), isobutyl aluminoxane, t-butyl aluminoxane, or mixtures thereof. In other non-limiting aspects, the aluminoxane can comprise, consist essentially of, or consist of, methylaluminoxane (MAO); alternatively, ethylaluminoxane; alternatively, modified methylaluminoxane (MMAO); alternatively, n-propylaluminoxane; alternatively, iso-propyl-aluminoxane; alternatively, n-butylaluminoxane; alternatively, sec-butylaluminoxane; alternatively, iso-butylaluminoxane; alternatively, t-butyl aluminoxane; alternatively, 1-pentyl-aluminoxane; alternatively, 2-pentylaluminoxane; alternatively, 3-pentyl-aluminoxane; alternatively, iso-pentyl-aluminoxane; or alternatively, neopentylaluminoxane. If the aluminoxane is a modified methylaluminoxane (MMAO), the aluminoxane can comprise MMAO-3A, MMAO-12, or a mixture thereof, although not limited thereto.

In certain aspects, the catalyst composition may further comprise an alkylaluminum compound. For instance, catalyst compositions disclosed herein can further comprise a trialkylaluminum, an alkylaluminum halide, an alkylaluminum alkoxide, or any combination thereof. In some aspects, the alkylaluminum compound can be a trialkylaluminum, an alkylaluminum halide, or any combination thereof. In other aspects, the alkylaluminum compound can be a trialkylaluminum; alternatively, an alkylaluminum halide; or alternatively, an alkylaluminum alkoxide.

In an aspect, each alkyl group of an aluminoxane (and/or alkylaluminum compound) independently can be a C₁ to C₂₀ alkyl group; alternatively, a C₁ to C₁₀ alkyl group; or alternatively, a C₁ to C₆ alkyl group. In an aspect, each alkyl group of an aluminoxane and/or alkylaluminum compound independently can be a methyl group, an ethyl group, a propyl group, a butyl group, a pentyl group, a hexyl group, a heptyl group, or an octyl group; alternatively, a methyl group, an ethyl group, a butyl group, a hexyl group, or an octyl group. In some aspects, each alkyl group of an aluminoxane and/or alkylaluminum compound can be a methyl group, an ethyl group, an n-propyl group, an n-butyl group, an iso-butyl group, an n-hexyl group, or an n-octyl group; alternatively, a methyl group, an ethyl group, an n-butyl group, or an iso-butyl group; alternatively, a methyl group; alternatively, an ethyl group; alternatively, an n-propyl group; alternatively, an n-butyl group; alternatively, an iso-butyl group; alternatively, an n-hexyl group; or alternatively, an n-octyl group.

In an aspect, each halide of any alkylaluminum halide disclosed herein independently can be chloride, bromide, or iodide. In some aspects, each halide of any alkylaluminum halide disclosed herein can be chloride or bromide; or alternatively, chloride.

In an aspect, each alkoxide group of any alkylaluminum alkoxide disclosed herein independently can be a C₁ to C₂₀, a C₁ to C₁₀, or a C₁ to C₆ alkoxy group. In an aspect, each alkoxide group of any alkylaluminum alkoxide disclosed herein independently can be a methoxy group, an ethoxy group, a propoxy group, a butoxy group, a pentoxy group, a hexoxy group, a heptoxy group, or an octoxy group; alternatively, a methoxy group, an ethoxy group, a butoxy group, a hexoxy group, or an octoxy group. In some aspects, each alkoxide group of any alkylaluminum alkoxide disclosed herein independently can be a methoxy group, an ethoxy group, an n-propoxy group, an n-butoxy group, an iso-butoxy group, an n-hexoxy group, or an n-octoxy group; alternatively, a methoxy group, an ethoxy group, an n-butoxy group, or an iso-butoxy group; alternatively, a methoxy group; alternatively, an ethoxy group; alternatively, an n-propoxy group; alternatively, an n-butoxy group; alternatively, an iso-butoxy group; alternatively, an n-hexoxy group; or alternatively, an n-octoxy group.

The relative amount of the aluminoxane activator (and any optional alkylaluminum) versus that of the transition metal complex is not particularly limited. Nonetheless, molar ratios of aluminum:transition metal in the catalyst compositions (and processes for preparing activated catalyst compositions and oligomerization processes utilizing the catalyst compositions) disclosed herein can range from 10:1 to 5,000:1, such as from 50:1 to 3,000:1, from 75:1 to 2,000:1, from 100:1 to 2,000:1, or from 100:1 to 1,000:1, and the like. If, for example, more than one transition metal complex and/or more than one aluminum compound are employed (e.g., aluminoxane and alkylaluminum), these ratios are based on the total moles of the respective transition metal complexes and aluminum compounds.

Catalyst compositions disclosed herein can comprise a solid support upon which the heteroatomic ligand transition metal complexes can be loaded or impregnated. Generally, the solid support can be any support that retains the activated catalyst compound in a solid state under oligomerization reaction conditions. In certain aspects, the solid support can comprise a solid oxide, a molecular sieve, diatomaceous earth, a zeolite, a carbon material (e.g., activated carbon), and the like, as well as any combination thereof.

Solid oxides that may be suitable as solid supports can include alumina (e.g., gamma alumina), silica, zirconia, silica-alumina, silica-zirconia, silica-coated alumina and the like. The solid oxide can encompass oxide materials such as silica, “mixed oxide” compounds thereof such as silica-titania, and combinations or mixtures of more than one solid oxide material. Mixed oxides such as silica-titania can be single or multiple chemical phases with more than one metal combined with oxygen to form the solid oxide. Examples of mixed oxides that can be used as the solid oxide include, but are not limited to, silica-alumina, silica-coated alumina, silica-titania, silica-zirconia, alumina-titania, alumina-zirconia, zinc-aluminate, alumina-boria, silica-boria, aluminum phosphate, aluminophosphate, aluminophosphate-silica, titania-zirconia, and the like, or a combination thereof. In some aspects, the solid oxide support can comprise silica, silica-alumina, silica-coated alumina, silica-titania, silica-titania-magnesia, silica-zirconia, silica-magnesia, silica-boria, aluminophosphate-silica, and the like, or any combination thereof. Silica-coated aluminas are encompassed herein; such oxide materials are described in, for example, U.S. Pat. Nos. 7,884,163 and 9,023,959.

The percentage of each oxide in a mixed oxide can vary depending upon the respective oxide materials. As an example, a silica-alumina (or silica-coated alumina) typically has an alumina content from 5 wt. % to 95 wt. %. According to one aspect, the alumina content of the silica-alumina (or silica-coated alumina) can be from 5 wt. % alumina 50 wt. % alumina, or from 8 wt. % to 30 wt. % alumina. In another aspect, high alumina content silica-aluminas (or silica-coated aluminas) can be employed, in which the alumina content of these materials typically ranges from 60 wt. % alumina to 90 wt. % alumina, or from 65 wt. % alumina to 80 wt. % alumina.

If the solid support is a carbon material, the solid support can comprise a carbon black, an activated carbon, a vulcanized carbon, a carbon nanomaterial (e.g., carbon nanotubes, carbon nanofibers), and the like, as well as any combination thereof.

The solid support (or the supported catalyst) can be characterized by any suitable BET surface area, pore volume, and average particle size. BET surface areas are determined using the BET nitrogen adsorption method of Brunauer et al., J. Am. Chem. Soc., 60, 309 (1938). While not limited thereto, the BET surface area often ranges from 2 to 1500 m²/g or from 50 to 900 m²/g, and more often, from 100 to 700 m²/g, from 150 to 350 m²/g, from 200 to 550 m²/g, from 200 to 350 m²/g, or from 250 to 450 m²/g. Pore volumes are determined in accordance with Halsey, G. D., J. Chem. Phys. (1948), 16, 931, and typically range from 0.2 to 5 mL/g. Illustrative and non-limiting ranges for the pore volume include from 0.2 to 2 mL/g, from 0.3 to 4 mL/g, from 0.3 to 3 mL/g, from 0.3 to 2 mL/g, from 0.5 to 2.5 mL/g, or from 1 to 3 mL/g.

The solid support (or the supported catalyst) can have any suitable shape or form, and such can depend on the type of oligomerization process/reactor (e.g., stirred tank reactor, loop slurry reactor, or fixed bed (or packed bed) reactor). Illustrative and non-limiting shapes and forms include powder, round or spherical (e.g., a sphere), ellipsoidal, pellet, bead, cylinder, granule (e.g., regular and/or irregular), trilobe, quadrilobe, ring, wagon wheel, monolith, and the like, as well as any combination thereof. In some aspects, the support or catalyst has a relatively small particle size, typically quantified by the d50 particle size, or median particle size, which refers to the particle size for which 50% of the sample by weight has a smaller size and 50% of the sample has a larger size. Particle size measurements are determined by laser diffraction in accordance with ISO 13320. In one aspect, the d50 average particle size of the solid support (or the supported catalyst) can be in a range from 5 to 500 μm, while in another aspect, the d50 particle size can range from 15 to 100 μm, and in another aspect, the d50 particle size can range from 25 to 400 μm, and in yet another aspect, the d50 particle size can range from 25 to 300 μm, and in still another aspect, the d50 particle size can range from 50 to 250 μm.

In other aspects, the solid support (or the supported catalyst) can be in the form of pellets or beads—and the like—having an average size ranging from 1/16 inch to ½ inch, or from ⅛ inch to ¼ inch, although not limited thereto. As noted above, the size of the support/catalyst particles can be varied to suit the particular oligomerization process and reactor system.

The amount of transition metal on the solid support is not particularly limited, but often falls within a range from 0.001 to 10 wt. % of the transition metal based on the total weight of the transition metal and the solid support. More often, the transition metal content can be in a range from 0.001 to 1 wt. %; alternatively, from 0.001 to 0.5 wt. %; alternatively, from 0.005 to 5 wt. %; alternatively, from 0.005 to 0.5 wt. %; alternatively, from 0.005 to 0.25 wt. %; alternatively, from 0.01 to 1 wt. %; alternatively, from 0.01 to 0.25 wt. %; or alternatively, from 0.01 to 0.05 wt. %. It is believed that lower metal loadings (e.g., less than or equal to 0.5 wt. %, 0.25 wt. %, or 0.05 wt. %) provide oligomerization activity and selectivity that is most comparable to that of homogeneous catalyst systems.

### Preparation of Heterogenous Catalyst Compositions

Processes for preparing an activated heterogenous catalyst composition consistent with aspects disclosed herein can comprise (a) contacting a heteroatomic ligand transition metal complex and an activator to form an activated catalyst, (b) contacting the activated catalyst and the solid support to form a supported catalyst, and (c) drying the supported catalyst to form the heterogenous catalyst composition. The heteroatomic ligand transition metal complex can have any of Formula (I), Formula (II), or Formula (III) as described above. If desired, the supported catalyst can be formed within the oligomerization reactor.

Forming the activated catalyst can be achieved under any suitable conditions. For instance, the heteroatomic ligand transition metal complex can be contacted with the activator at an activation temperature is in a range from −20 °C to 80 °C, and more often, from 0 °C to 80 °C, from 0 °C to 60 °C, from 0 °C to 50 °C, or from 0 °C to 40 °C. Generally, the heteroatomic ligand transition metal complex and the activator are contacted in a hydrocarbon solvent by mixing a solution or slurry of the heteroatomic ligand transition metal complex in a hydrocarbon solvent with a solution or slurry of the activator in the same or a different hydrocarbon solvent. Suitable hydrocarbon solvents can include, for example, aliphatic hydrocarbons (e.g., pentane, hexane, heptane, octane, decane, and combinations thereof) and aromatic hydrocarbons (e.g., benzene, toluene, xylene(s), ethylbenzene, and combinations thereof).

Once the heteroatomic ligand transition metal complex and the activator have been contacted to form the activated catalyst, the activated catalyst can be loaded or impregnated onto the solid support by contacting the activated catalyst with the solid support to form the supported catalyst. In certain aspects, the solid support can have a relatively low moisture content to ensure that the activated catalyst retains its activity upon contact with the solid support. Although not limited thereto, the solid support can have a moisture content of less than or equal to 2 wt. %, less than or equal to 1.5 wt. %, less than or equal to 1 wt. %, or less than or equal to 0.5 wt. %.

The activated catalyst can be contacted with the solid support in any suitable manner. A particularly suitable technique is to use incipient wetness impregnation methodology, which comprises combining the solid support with a solution of the activated catalyst in a hydrocarbon solvent. Generally, incipient wetness impregnation can comprise contacting the activated catalyst in the hydrocarbon solvent with the solid support in sufficient amount to load the solvated catalyst into the pores of the solid support, but not to exceed the total pore volume of the solid support by a substantial amount such that the solid support may become oversaturated with solvent. Thus, the amount of the activated catalyst (e.g., wt. % of transition metal based on weight of the solid support) can be quantified relative to the total pore volume of the solid support. In certain aspects, contacting the activated catalyst and the solid support can comprise contacting an amount of the activated catalyst less than 500%, less than 200%, less than 100%, or less than 50% of a total pore volume of the solid support. Generally, the total pore volume of the solid oxide (or the supported catalyst) falls within a range from 0.2 to 5 mL/g, but is not limited thereto. Illustrative and non-limiting ranges for the total pore volume include from 0.2 to 2 mL/g, from 0.3 to 4 mL/g, from 0.3 to 3 mL/g, from 0.3 to 2 mL/g, from 0.5 to 2.5 mL/g, or from 1 to 3 mL/g.

The supported catalyst can then be dried to remove residual hydrocarbon solvent. Drying the supported catalyst can comprise any suitable technique or any technique disclosed herein. For instance, any excess liquid can be removed from the supported catalyst (e.g., draining, filtering, etc.), and the wet supported catalyst can be subjected to a wide range of drying times, drying temperatures, and drying pressures. For example, the drying time can range from 15 min to 48 hours, from 30 min to 24 hours, or from 1 to 12 hours, and the drying temperature can range from 0 °C to 300 °C, from 20 °C to 200 °C, or from 100 °C to 175 °C. The drying pressure can be at or around atmospheric pressure, but in many instances, the drying step can be conducted under vacuum conditions at any suitable sub-atmospheric pressure, such as less than 100 torr (13.3 kPa), less than 50 (6.67 kPa) torr, or less than 10 torr (1.33 kPa). Conditions for drying the supported catalyst prepared by incipient wetness impregnation may be relatively mild due to the relatively small amount of solvent present.

Surprisingly, it was found that the heterogenous catalyst compositions prepared in the manner described above retained a catalytic activity for ethylene oligomerization similar to that of otherwise identical homogenous catalyst systems as shown in the Examples that follow.

### Oligomerization Processes

Aspects of this disclosure are directed to ethylene oligomerization processes, the production of an oligomer product, and the isolation and recovery of certain carbon number oligomers. One such oligomerization process can comprise (or consist essentially of, or consist of) (I) contacting ethylene, any heterogenous catalyst composition disclosed herein, and optionally hydrogen, in an oligomerization reactor, (II) forming an oligomer product in the oligomerization reactor, the oligomer product comprising C₄ to C₂₀ ethylene oligomers, and (III) discharging an effluent stream from the oligomerization reactor, the effluent stream comprising unreacted ethylene and the oligomer product. In certain aspects, in the effluent stream, the unreacted ethylene can be a liquid and the oligomer product can be a liquid. Alternatively, the unreacted ethylene can be a gas and the oligomer product also can be a gas in the effluent stream. In still further aspects, the unreacted ethylene can be a gas, and the oligomer product can be a liquid.

Referring now to step (I), ethylene and a solid activated catalyst composition formed as described above can be contacted in an oligomerization reactor under oligomerization conditions. Ethylene and the catalyst composition (and hydrogen, if used) can be combined in any order or sequence and introduced into the oligomerization reactor separately or in any combination. Due to the heterogenous nature of the catalyst composition comprising a solid support, the ethylene may be introduced into the oligomerization reactor as a gas in one aspect, and as liquid in another aspect, and contacted with the activated catalyst on a solid support to form a liquid oligomer product. In such aspects, the liquid oligomer product may be easily separated from the effluent stream discharged from the oligomerization reactor, and from unreacted ethylene and hydrogen (if any). The unreacted ethylene can then be recycled for use in further oligomerization processes, and any residual solid catalyst can be easily separated from the effluent stream by filtration or the like, depending upon reactor configuration.

Forming the oligomer product in the oligomerization reactor can be conducted at any suitable oligomerization temperature and pressure. Often, the oligomer product can be formed at a minimum temperature of 0 °C, 15 °C, 20 °C, 30 °C, 40 °C, or 50 °C; additionally or alternatively, at a maximum temperature of 165 °C, 150 °C, 130 °C, 115 °C, 100 °C, 80 °C, or 55 °C. Generally, the oligomerization temperature at which the oligomer product is formed can be in a range from any minimum temperature disclosed herein to any maximum temperature disclosed herein. Accordingly, suitable non-limiting ranges can include the following: from 0 to 165, from 0 to 115, from 15 to 115, from 15 to 100, from 20 to 130, from 20 to 80, from 20 to 55, from 30 to 150, from 40 to 100, from 40 to 80, from 50 to 150, or from 50 to 80 °C. Other appropriate oligomerization temperatures and temperature ranges are readily apparent from this disclosure.

The oligomer product can be formed at a minimum pressure (or ethylene partial pressure) of 15 psig (103 kPa), 50 psig (344 kPa), 100 psig (689 kPa), or 200 psig (1.4 MPa); additionally, or alternatively, at a maximum pressure (or ethylene partial pressure) of 4,000 psig (27.6 MPa), 2,000 psig (13.8 MPa), 1,500 psig (10.3 MPa), or 250 psig (1.5 MPa). Generally, the oligomerization pressure (or ethylene partial pressure) at which the oligomer product is formed can be in a range from any minimum pressure disclosed herein to any maximum pressure disclosed herein. Accordingly, suitable non-limiting ranges can include the following: from 15 psig (103 kPa) to 4,000 psig (27.6 MPa), from 15 psig (103 kPa) to 1,500 psig (10.3 MPa), from 15 psig (103 kPa) to 250 psig (1.5 MPa), from 50 psig (344 kPa) to 2,000 psig (13.8 MPa), from 50 psig (344 kPa) to 1,500 psig (10.3 MPa), from 100 psig (689 kPa) to 1,500 psig (10.3 MPa), or from 200 psig (1.4 MPa) to 1,500 psig (10.3 MPa). Other appropriate oligomerization pressures (or ethylene partial pressures) are readily apparent from this disclosure.

In certain aspects, hydrogen is not present in step (I) of the oligomerization process. Alternatively, when used, hydrogen can be fed directly to the reactor, or hydrogen can be combined with an ethylene feed prior to the reactor. In the reactor, the hydrogen partial pressure can be at least 1 psig (6.9 kPa), 5 psig (34 kPa), 10 psig (69 kPa), 25 psig (172 kPa), or 50 psig (345 kPa); additionally or alternatively, a maximum hydrogen partial pressure of 2000 psig (13.8 MPa), 1750 psig (12.1 MPa), 1500 psig (10.3 MPa), 1250 psig (8.6 MPa), 1000 psig (6.9 MPa), 750 psig (5.2 MPa), 500 psig (3.4 MPa), or 400 psig (2.8 MPa). Generally, the hydrogen partial pressure can range from any minimum hydrogen partial pressure disclosed herein to any maximum hydrogen partial pressure disclosed herein. Therefore, suitable non-limiting ranges for the hydrogen partial pressure can include the following ranges: from 1 psig (6.9 kPa) to 2000 psig (13.8 MPa), from 1 psig (6.9 kPa) to 1750 psig (12.1 MPa), from 5 psig (34 kPa) to 1500 psig (10.3 MPa), from 5 psig (34 kPa) to 1250 psig (8.6 MPa), from 10 psig (69 kPa) to 1000 psig (6.9 MPa), from 10 psig (69 kPa) to 750 psig (5.2 MPa), from 10 psig (69 kPa) to 500 psig (3.5 MPa), from 25 psig (172 kPa) to 750 psig (5.2 MPa), from 25 psig (172 kPa) to 500 psig (3.4 MPa), from 25 psig (172 kPa) to 400 psig (2.8 MPa), or from 50 psig (345 kPa) to 500 psig (3.4 MPa). Other appropriate hydrogen partial pressures in the reactor for the formation of the oligomer product are readily apparent from this disclosure.

Referring now to step (II), an oligomer product is formed in the oligomerization reactor, and the oligomer product comprises C₄ to C₂₀ ethylene oligomers. The oligomerization reactor in which the oligomer product is formed in step (II) (and in which the components in step (I) are contacted) can comprise any suitable reactor. Non-limiting examples of reactor types can include a stirred tank reactor, a plug flow reactor, or any combination thereof; alternatively, a fixed bed reactor, a continuous stirred tank reactor, a loop reactor, a tubular reactor, a recycle reactor, or any combination thereof. For instance, in one aspect, the oligomerization reactor can comprise a continuous stirred tank reactor, while in another aspect, the oligomerization reactor can comprise a loop slurry reactor, and in yet another aspect, the oligomerization reactor can comprise a fixed bed reactor. In the fixed bed arrangement, the ethylene feed in step (I) contacts a fixed bed of the solid catalyst composition in the oligomerization reactor. In some aspects, there can be more than one reactor in series or in parallel, and including any combination of reactor types and arrangements. Moreover, the oligomerization process used to form the oligomer product can be a continuous process or a batch process, or any reactor or vessel utilized in the process can be operated continuously or batchwise.

Among other constituents, the effluent stream contains the oligomer product, which can comprise butenes, hexenes, and octenes, as well as other C₄₊ linear alpha olefins. The amount of C₄ to C₈ or C₆ to C₅ oligomer in the oligomer product typically can fall within a range from 5 to 99 mol %, based on the total amount of oligomers in the oligomer product. In an aspect, the amount of octenes—based on the total weight of oligomers in the oligomer product—can be in a range from 5 to 85 mol %, from 10 to 90 mol %, from 20 to 99 mol %, from 30 to 95 mol %, from 40 to 95 mol %, from 40 to 90 mol %, from 20 to 90 mol %, from 30 to 87.5 mol %, from 30 to 85 mol %, from 40 to 87.5 mol %, from 40 to 85 mol %. Additionally, or alternatively, the amount of hexenes—based on the total weight of oligomers in the oligomer product—can be from 10 to 75 mol %, from 15 to 65 mol %, from 20 to 60 mol %, from 25 to 55 mol %, or from 30 to 50 mol % hexenes.

Optionally, the disclosed oligomerization processes—after step (III)—can further comprise a step of separating (all or any portion of) the catalyst composition and (all or any portion of) the oligomer product (or a step of separating (all or any portion of) the catalyst composition and (all or any portion of) the effluent stream), by any suitable solid-liquid separations technique. For instance, and not limited thereto, the step of separating can employ draining, decanting, pressing, centrifuging, filtering, sedimenting, and the like, as well as any combination thereof.

Optionally, the disclosed oligomerization processes—after step (III)—can further comprise a step of separating or isolating (all or any portion of) one or more Cₙ (e.g., butenes, hexenes, octenes, decenes) oligomer streams from the oligomer product. One such technique for isolating (all or any portion of) the oligomer product can comprise a step of removing (all or any portion of) the unreacted ethylene from the effluent stream. In these and other aspects, various suitable separation or isolation steps can be employed, as would be recognized by those of skill in the art. In an aspect, such separation or isolation steps can include one or more batch or continuous flash processes (or evaporation processes), one or more batch or continuous distillation processes, and combinations thereof. For instance, the effluent stream can be subjected to flashing or evaporation under reduced pressure (as compared to the pressure in the oligomerization reactor) thereby removing (all or any portion of) the unreacted ethylene from the effluent stream via the flashing or evaporating under reduced pressure. While not limited thereto, the reduced pressure for flashing or evaporating can range from 5 to 15 psig.

Distillation processes to fractionate the oligomer product and to isolate various carbon number streams (e.g., C₆ olefins, C₈ olefins, or both C₆ to C₈ olefins) can be performed at atmospheric or any suitable elevated pressure or sub-atmospheric pressure. Suitable sub-atmospheric pressures can include, but are not limited to, less than 100 torr (13.3 kPa), less than 50 (6.67 kPa) torr, less than 10 torr (1.33 kPa), or less than 5 torr (0.67 kPa). The conditions that are used to fractionate the oligomer product can be varied based on the desired carbon number streams and the identity and/or quantity of the particular carbon number oligomer(s) to be isolated.

In certain aspects, the separated (or isolated) oligomer streams individually can contain any suitable amount of the isolated oligomer, such as at least 96, at least 97, at least 98, or at least 99 mol % Cₙ oligomer(s), based on the total weight of the Cₙ stream. For instance, in certain aspects, the separated (or isolated) C₈ stream can contain any suitable amount of octene(s), such as at least 96, at least 97, at least 98, or at least 99 mol % octene(s), based on the total weight of the C₈ stream.

Generally, a vast majority of these Cₙ streams are desirable α-olefin products, e.g., 1-hexene and 1-octene, for instance. The C₆ stream can contain, for example, at least 90 mol % 1-hexene, and more often at least 92.5 mol %, at least 95 mol %, at least 97.5 mol %, at least 98 mol %, at least 98.5 mol %, or at least 99 mol % 1-hexene, based on the total weight of the hexene(s) in the C₆ stream. Likewise, the C₈ stream can contain, for example, at least 95 mol % 1-octene, and more often, at least 96 mol %, at least 96.5 mol %, at least 97 mol %, at least 97.5 mol %, at least 98 mol %, or 98.5 mol % 1-octene, based on the total weight of the octene(s) in the C₈ stream.

The oligomer product can be formed at any desired ethylene conversion (or single pass ethylene conversion). The oligomer product can be formed at a minimum ethylene conversion (or single pass ethylene conversion) of 5, 20, 30, 35, 40, 45, or 55 mol %; additionally or alternatively, a maximum ethylene conversion (or single pass ethylene conversion) of 99, 95, 90, 80, 75, 70, or 65 mol %. Generally, the oligomer product can be formed at an ethylene conversion (or single pass ethylene conversion) that can range from any minimum ethylene conversion (or single pass ethylene conversion) disclosed herein to any maximum ethylene conversion (or single pass ethylene conversion) disclosed herein. In some non-limiting aspects, the oligomer product can be formed at an ethylene conversion (or single pass ethylene conversion) ranging from 5 to 95 mol %, from 20 to 95 mol %, from 30 to 90 mol %, from 40 to 80 mol %, from 50 to 70 mol %, or from 50 to 65 mol %. These conversions are based on the amount of ethylene entering the reactor and the amount of unreacted ethylene in the effluent stream. Other ethylene conversion (or single pass ethylene conversion) ranges that can be utilized are readily apparent to those skilled in the art with the aid of this disclosure.

In certain aspects, the productivity of the heterogenous catalyst composition can be relatively high. For instance, the productivity of the heterogenous catalyst compositions disclosed herein (in kg oligomer product/g transition metal, in kg oligomer product/g transition metal/hr, or in kg oligomer product/g Al), can be within 10%, within 25%, or within 40% of that of an otherwise identical homogenous catalyst composition comprising the same heteroatomic ligand transition metal complex and activator, and under identical oligomerization conditions. For these productivity measurements, the amount of the oligomer product is the total amount of C₄ to C₂₀ olefins in the oligomer product.

An unexpected and beneficial attribute of the disclosed heterogenous catalyst compositions is that they may be used more than once, for instance, they may be reused in several oligomerization cycles. Thus, the solid catalyst composition can be retained within the oligomerization reactor throughout the oligomerization process, and repeated and/or continuous runs of ethylene oligomerization can be conducted without having to change out the catalyst. In certain aspects of this disclosure, a (first) oligomerization reaction can be conducted according to steps (I)-(III) described above, and the process can further comprise a second oligomerization process in which the catalyst composition is reused. This second oligomerization process can comprise (IV) optionally, regenerating the catalyst composition within the oligomerization reactor, (V) contacting ethylene and optionally hydrogen in the oligomerization reactor with the catalyst composition, (VI) forming a second oligomer product in the oligomerization reactor, the second oligomer product comprising C₄ to C₂₀ ethylene oligomers, and (VII) discharging a second effluent stream from the oligomerization reactor, the second effluent stream comprising a second unreacted ethylene and the second oligomer product. Advantageously, (all or any portion of) the ethylene in step (V) comprises the (all or any portion of) the unreacted ethylene recovered from step (III).

Further aspects can comprise additional subsequent oligomerization processes as may be feasible according to the longevity of the catalyst composition within the oligomerization reactor where acceptable productivity and/or selectivity to α-olefins remains. In certain aspects, the oligomerization process can proceed over many oligomerization cycles without restoring or replacing the catalyst composition within the oligomerization reactor, as demonstrated in the Examples that follow.

Nonetheless, it can be beneficial to regenerate the catalyst composition, as needed, to ensure acceptable productivity and/or selectivity. Ordinarily, the step of regenerating the catalyst composition occurs within the oligomerization reactor, often following a first oligomerization process. In such aspects, the first oligomerization can be a batch process or a continuous process, each of which is terminated at a particular reaction time. The reaction time can be predetermined by an amount of the ethylene reactant and the productivity of the catalyst composition. Alternatively, a minimum threshold value of the catalyst composition productivity and/or selectivity can be monitored in a continuous process and terminated upon reaching said threshold value.

Once terminated, the catalyst composition can be regenerated for use in subsequent oligomerization processes. In certain aspects, regenerating the catalyst composition can comprise purging the catalyst composition with nitrogen gas. The nitrogen gas used for regenerating the catalyst composition will typically have a very low moisture content of less than 100 ppm, and more often, less than 50 ppm, less than 10 ppm, less than 5 ppm, less than 3 ppm, or less than 1 ppm (by weight).

In this manner, the activity, productivity, and/or selectivity of the catalyst composition may be restored to or near its original values observed in step (II) of the oligomerization processes described herein. Thus, in second oligomerizations (e.g., as observed in step (VI) of the processes disclosed herein), an activity, productivity, and/or selectivity of the catalyst composition can be in a range from 20% to 200%, from 20% to 150%, or from 20% to 95%, of that of the respective activity, productivity, and/or selectivity of the catalyst composition in step (II) of the oligomerization process. Additionally or alternatively, a single pass ethylene conversion in steps (V)-(VII) can be within 10%, within 25%, or within 40%, of that of steps (I)-(III), utilizing the same catalyst composition (with or without an intermediate regeneration of the catalyst composition) under the same oligomerization reaction conditions.

### EXAMPLES

The disclosure is further illustrated by the following examples, which are not to be construed in any way as imposing limitations to the scope of this disclosure. Various other aspects, modifications, and equivalents thereof which, after reading the description herein, can suggest themselves to one of ordinary skill in the art without departing from the spirit of the present disclosure or the scope of the appended claims.

### Examples 1-13. Iron Catalyst Compositions for Ethylene Oligomerization

Iron complexes having structures consistent with Formula (I) were prepared to exemplify the characteristics of the improved supported catalyst compositions and their use within ethylene oligomerization processes. Various catalyst compositions were evaluated for their effectiveness in oligomerization processes to selectively produce light α-oligomers, as summarized in Examples 1-13 below.

Activated catalyst compounds were loaded onto solid supports by either wet impregnation or incipient wetness impregnation method. Generally, wet impregnation (WI) methods included preparing a solution of the iron complex activated by aluminoxane and combining the solid support and the solution with stirring to create a slurry. The solvent was then removed by vacuum to yield a dry powder. Alternatively, for examples that employed incipient wetness impregnation (IWI), a solution of the activated catalyst compound was added not in excess of the pore volume of the solid support such that no additional solvent was filtered off from the resulting mixture. The powders were dried under vacuum.

### Comparative Example 1: Ethylene Oligomerizations Using a Homogenous Iron Catalyst Composition for Ethylene Oligomerization

A catalyst composition comprising an α-diimine iron complex (Fe-1, structure shown below) was prepared by first dissolving Fe-1 (9.05 μmol) in toluene. MMAO (500 eq.) was then added to the solution to activate Fe-1 catalyst. No solid support was added, and the activated catalyst composition was used in its solution phase for ethylene oligomerization in a customized VCO reactor (with maximum volume of 9 mL, normal operation volume ≤5 mL) for 1 h. The customized VCO reactor was designed using a custom made thick wall 316 stainless-steel reactor bottom with a glass or Teflon cup inside to hold the reaction solution, which had a pressure limit of 2,000 psi at room temperature. The reactor bottom was connected to the top part of the reactor using a 1-inch Swagelok VCO O-ring face seal fitting. A Teflon O-ring was used for high temperature reactions (>100 °C) or reactions with corrosive reagents, while a Viton O-ring was used for reactions at low temperatures. The top part of the reactor was connected to a ¼-inch straight ball valve via either Swagelok or NPT type reducing connectors. All reagents and a PTFE magnetic stir bar were assembled to the customized VCO reactor inside a nitrogen filled glovebox, and the assembly was sealed to protect the catalyst mixture from air and moisture before being removed from the glovebox. The reactor was then connected to the ethylene feeding line though the top of the straight ball valve via ¼-inch Swagelok connector, after purging out the remaining air in the tube. After setting the stir rate to 300 rpm, 200 psig of ethylene was charged into the reactor and maintained at 200 psig during the reaction using a Parr high pressure gas burette. The reaction was performed at room temperature without external heating or cooling. Products were analyzed by gas chromatography, using an internal standard, and the results are shown in Table I below.

### Examples 2-9: Ethylene Oligomerizations Using a Heterogenous Iron Catalyst Composition

For each of Examples 2-9, a Fe-1 catalyst composition similar to that prepared in Comparative Example 1 was loaded onto a solid support to yield a heterogenous oligomerization catalyst composition. Unless otherwise indicated, approximately 9.05 μmol of the metal catalyst was used in all of the heterogenous examples (the transition metal weight percent was generally in the 0.006 wt. % to 0.26 wt. % range). The γ-Al₂O₃ had a pore volume of 0.8-1.2 mL/g and a BET surface area of 220-280 m²/g, and the zeolite in Example 5 had a d50 of less than 9 μm, a pore volume of 0.3 mL/g, and a BET surface area of 550-700 m²/g. Preparation methods and reaction conditions are summarized for each of Examples 2-9 in Table I below. In each of Examples 2-9, the heterogenous catalyst composition was loaded into a custom made batch reactor having a ½-inch 316 stainless steel tube catalyst chamber for ethylene oligomerization. During the entire process, the active catalysts were protected from air and moisture. The ethylene pressure was maintained constant (e.g., 200 psig) during the catalytic reaction by consistent feeding. No external heating or cooling were used to initiate the reaction, and the catalyst chamber temperature was monitored using a temperature probe. Below the catalyst chamber, a 316 stainless steel liquid trap (cooling to −15 °C) was used to collect the liquid products formed during the reaction.

Generally, as shown below, the heterogenous catalysts of Examples 2-9 showed a similar productivity with respect to C₄ to C₂₀ oligomers and selectivity to α-olefin products as Comparative Example 1 using a homogenous catalyst composition. Particularly, Examples 5-6 using an alumina solid support retained the high selectivity to α-olefin products. Additionally, the resulting product mixture was weighted significantly toward lighter C₄ and C₆ oligomer products.

The results of Examples 2-9 also provided insight into the effect of catalyst loading methods on the catalyst productivity and selectivity. The heterogenous catalyst for Examples 2-6 was prepared according to the wet impregnation procedure as described above, whereas for Examples 7-9, incipient wetness impregnation was used. Examples 6 and 7 were conducted under similar reaction conditions, however, Example 7 (IWI) demonstrated an improved productivity and lighter oligomer product distribution compared to Example 6 (WI), while maintaining selectivity toward α-olefin products.

### Examples 10-13. Heterogenous Catalyst Compositions Comprising Other α-Diimine Iron Complexes

Additional α-diimine iron complexes with bulkier substituents on the phosphine as well as the imine arene ring were prepared generally as described above. The structures for Fe-2 to Fe-5 are shown below (Ph=phenyl; Ac=acetate). Heterogenous catalyst compositions comprising each of the iron complexes were prepared by incipient wetness impregnation (IWI) of the complexes onto alumina. Each heterogenous catalyst composition was employed in ethylene oligomerization under conditions as described for Example 7 above. Results of the ethylene oligomerization for Examples 10-13 are also shown in Table I.

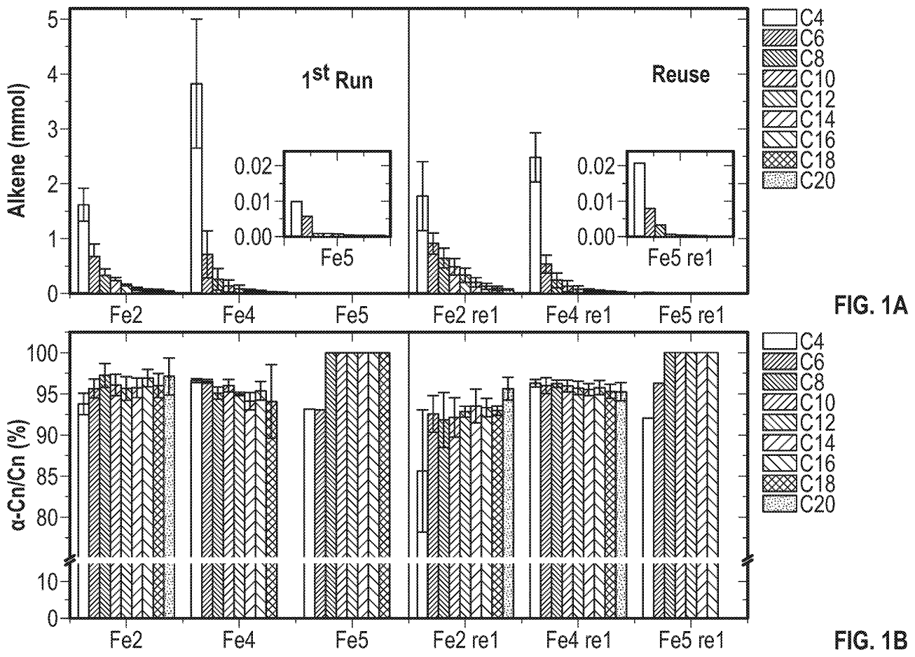

As for Examples 2-9, each of these catalyst compositions also demonstrated at least comparable productivity and selectivity to ethylene oligomerization using a homogenous catalyst composition (Example 1). Again, the light oligomers were strongly favored in the product mixture, comprising 71 mol % C₄ and C₆ oligomers in Example 11. Surprisingly, Example 12 demonstrated unexpected selectivity both with respect to C₄ and C₆ oligomers, which represented 89 mol % of the product mixture, while also producing 95 mol % α-oligomers. The comparison of Fe-2, Fe-4 and Fe-5 is shown in FIG. 1A and FIG. 1B.

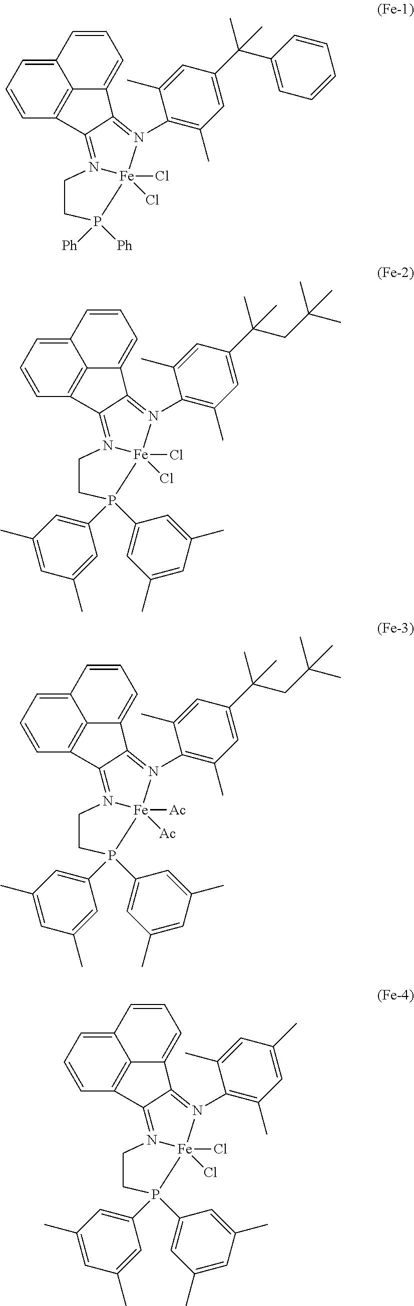

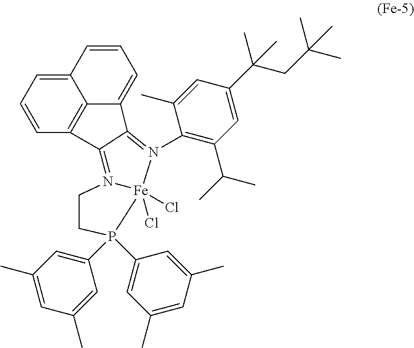

TABLE I
Summary of preparation and oligomer product characterization from Examples 1-11.

| | | Al activator | Support | | Reaction | Catalyst | α-C₄/C₄ | C₄/Cₙ | α-C₆/C₆ | C₆/Cₙ | C₄₋₆/Cₙ |
|---|---|---|---|---|---|---|---|---|---|---|---|
| Example | Catalyst | (equiv.) | (mass) | Reactor | time | loading | (%) | (mol %) | (%) | (mol %) | (mol %) |
| 1 | Fe-1 | MMAO-12 | in toluene | VCO | 1 h | N/A | 98 | 38 | 98 | 20 | 58 |
| | | (500 eq.) | | | | | | | | | |
| 2 | Fe-1 | MMAO-12 | Celite | Batch | 2 h | WI | 74 | 85 | 76 | 3 | 88 |
| | | (500 eq.) | (400 mg) | | | | | | | | |
| 3 | Fe-1 | MMAO-12 | Celite | Batch | 2 h | WI | 99 | 91 | >99 | 8 | 99 |
| | | (35 eq.) | (400 mg) | | | | | | | | |
| 4 | Fe-1 | MMAO-12 | Celite | Batch | 2 h | WI | 64 | 84 | 64 | 4 | 88 |
| | | (500 eq.) | (200 mg) | | | | | | | | |
| 5 | Fe-1 | MMAO-12 | Zeolite | Batch | 2 h | WI | 91 | 87 | 82 | 2 | 89 |
| | | (500 eq.) | (200 mg) | | | | | | | | |
| 6 | Fe-1 | MMAO-12 | γ-Al₂O₃ | Batch | 1 h | WI | 87 | 67 | 89 | 30 | 97 |
| | | (500 eq.) | (0.8 g) | | | | | | | | |
| 7 | Fe-1 | MMAO-12 | γ-Al₂O₃ | Batch | 1 h | IWI | 89 | 78 | 92 | 7 | 85 |
| | | (500 eq.) | (2.5 g) | | | | | | | | |
| 8 | Fe-1 | MMAO-12 | 3A sieves | Batch | 1 h | IWI | 89 | 58 | 89 | 12 | 70 |
| | | (500 eq.) | (8 g) | | | | | | | | |
| 9 | Fe-1 | MMAO-12 | 3A sieves | Batch | 1 h | IWI | 64 | 73 | 59 | 6 | 79 |
| | | (500 eq.) | (6 g) | | | | | | | | |
| 10 | Fe-2 | MMAO-12 | γ-Al₂O₃ | Batch | 1 h | IWI | 89 | 64 | 89 | 8 | 72 |
| | | (500 eq.) | (2.5 g) | | | | | | | | |
| 11 | Fe-2 | MMAO-3A | γ-Al₂O₃ | Batch | 1 h | IWI | 94 | 51 | 96 | 20 | 71 |
| | | (500 eq.) | (2.5 g) | | | | | | | | |
| 12 | Fe-4 | MMAO-3A | γ-Al₂O₃ | Batch | 1 h | IWI | 95 | 77 | 97 | 12 | 89 |
| | | (500 eq.) | (2.5 g) | | | | | | | | |
| 13 | Fe-5 | MMAO-3A | γ-Al₂O₃ | Batch | 1 h | IWI | 93 | 55 | 93 | 31 | 86 |
| | | (500 eq.) | (2.5 g) | | | | | | | | |

### Examples 14-29: Ethylene Oligomerization Processes Reusing Heterogenous Iron Catalyst Compositions

As described above, heterogenous catalyst compositions can provide for more efficient downstream processes, including separation of the oligomer product from the effluent stream. For instance, gaseous unreacted ethylene can be separated from liquid oligomer product in the product stream by evaporation under reduced pressure, and any residual solid catalyst may be removed from the liquid product by filtration, depending upon reactor configuration. Heterogenous catalyst compositions also may be reused and or regenerated within the reactor, such that subsequent oligomerization reactions do not need a separate preparation of the catalyst composition.

Examples 14-29 examine conditions of ethylene oligomerization for which the heterogenous catalyst system was reused for subsequent ethylene oligomerizations without replacing the catalyst system in the reactor.

### Example 14: Ethylene Oligomerization Processes without Intermediate Catalyst Regeneration

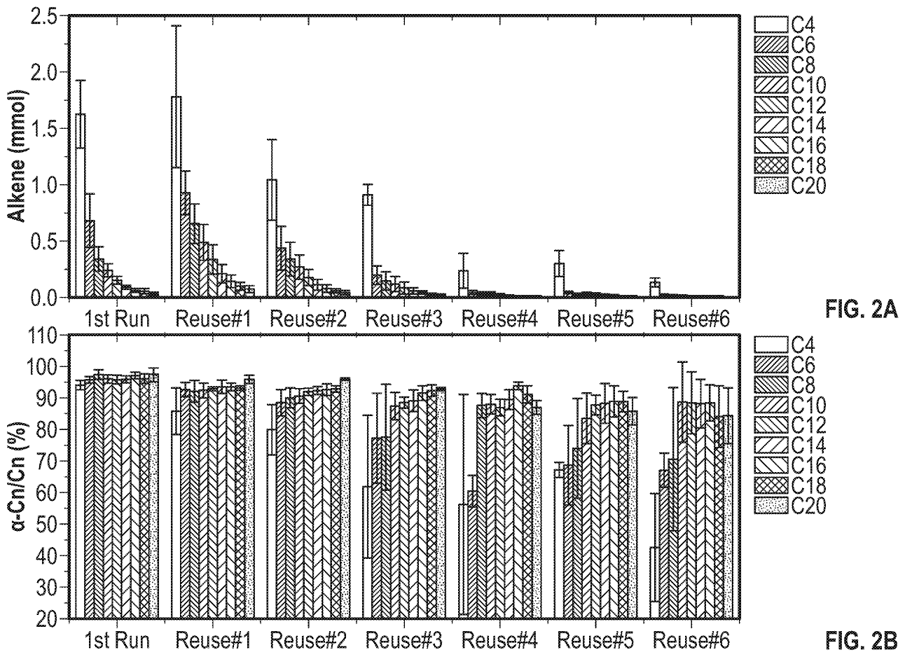

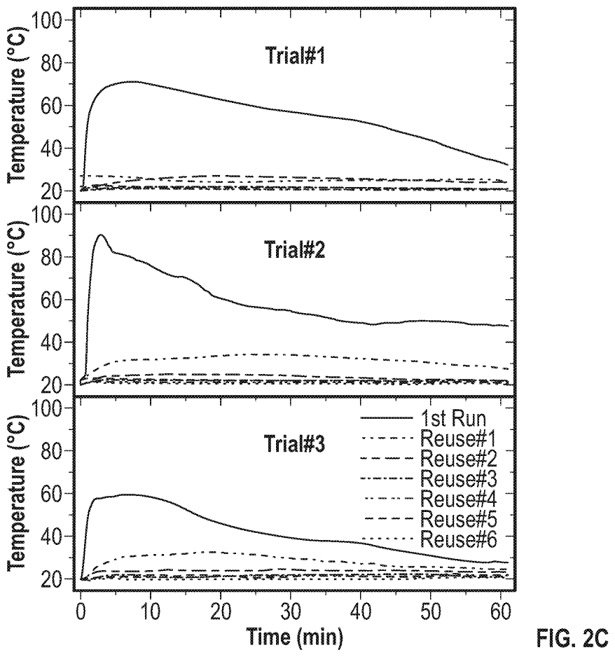

A series of seven consecutive ethylene oligomerizations were conducted using the Fe-2 heterogenous catalyst composition, generally as prepared and under the same reaction conditions as described above for Example 11. This experiment was conducted without intermediate catalyst regeneration between the subsequent oligomerization reactions. After each batch of oligomerization, the product stream was separated from the reactor by condensing into the liquid trap and the catalyst chamber was sealed after releasing the pressure. Then, the cooled liquid trap was removed, and the collected products were quantified. A new liquid trap was assembled, and the air was removed either via nitrogen purge or vacuum/N₂ refill cycles. After that, the reactor was filled with ethylene and then the catalyst chamber was opened again. The results of each run with respect to olefin productivity, selectivity, and reactor temperature are shown in FIGS. 2A-2C. Generally, it was shown that the catalyst was able to be reused several times without need for replacing the solid catalyst within the reactor. Each of the first three subsequent runs experienced a gradual and slight decrease in productivity; however, the selectivity to lighter α-olefins remained high in each of subsequent runs 1-3. A significant decrease in productivity and α-olefin selectivity was observed after the third subsequent oligomerization run. It was also observed that the reactor did not experience an exotherm and the temperature remained fairly constant during each of the subsequent oligomerizations as shown in FIG. 2C.

### Example 15: Ethylene Oligomerization Processes Using Catalyst Regenerated by Nitrogen Gas

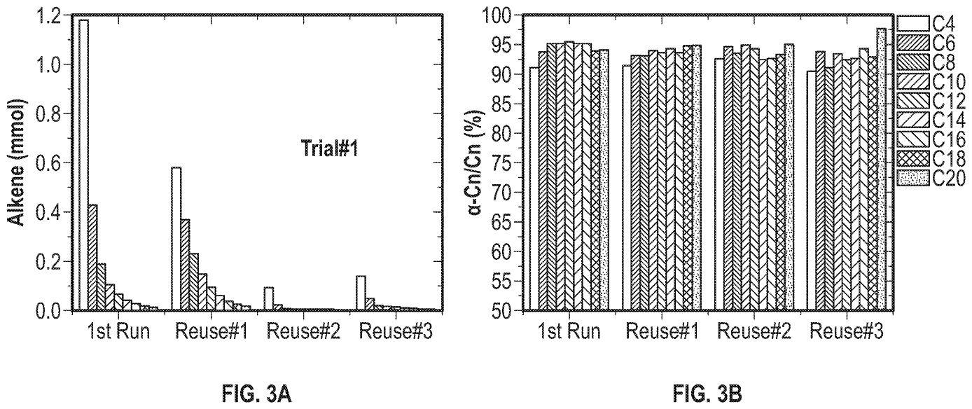

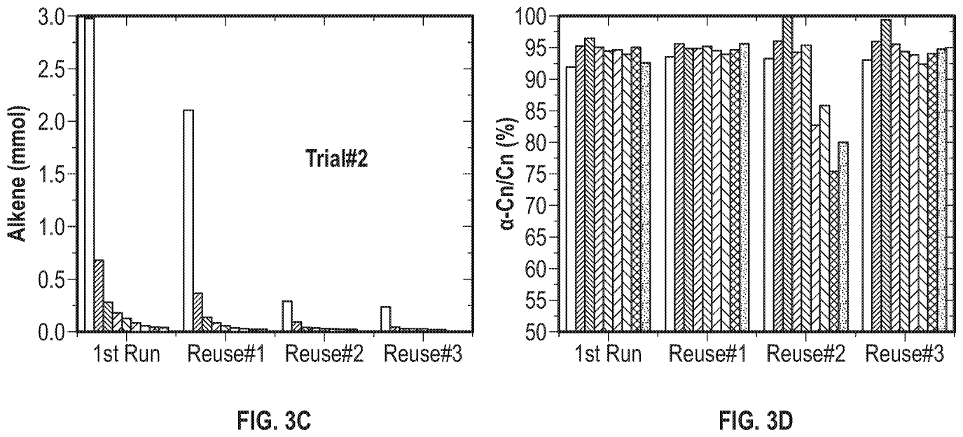

A series of four oligomerization reactions was performed generally as described in Example 12, using a house N₂ purge to the catalyst chamber prior to each oligomerization reaction. Results from Example 15 are shown in FIGS. 3A-3D. The selectivity to lighter α-oligomers was retained; however, it was observed that the decrease in activity in subsequent runs was less gradual, with significant decreases noted in the second reuse as opposed to the fourth.

### Example 16: Ethylene Oligomerization Processes Using Catalyst Regenerated by Low-Moisture Nitrogen Gas

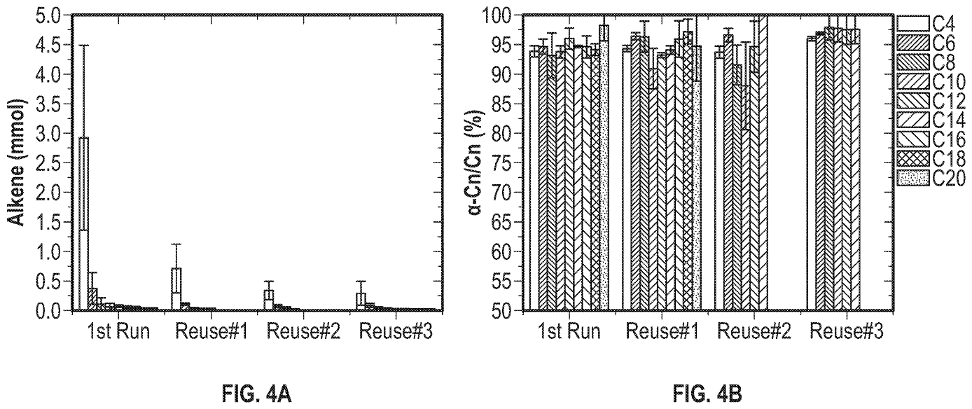

A series of four oligomerization reactions was performed generally as described in Example 15, except that a low moisture high purity N₂ source was used. As shown in FIG. 4B, the selectivity for light oligomers was even greater than in Example 15, with the product mixture heavily favoring 1-butene in greater than 5:1 excess over 1-hexene (Table II).

### Example 17: Ethylene Oligomerization Processes Using Reduced Reaction Time

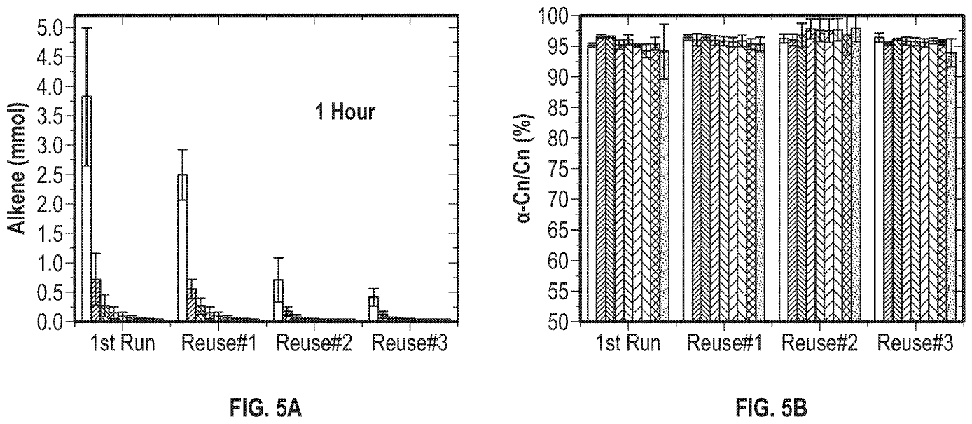

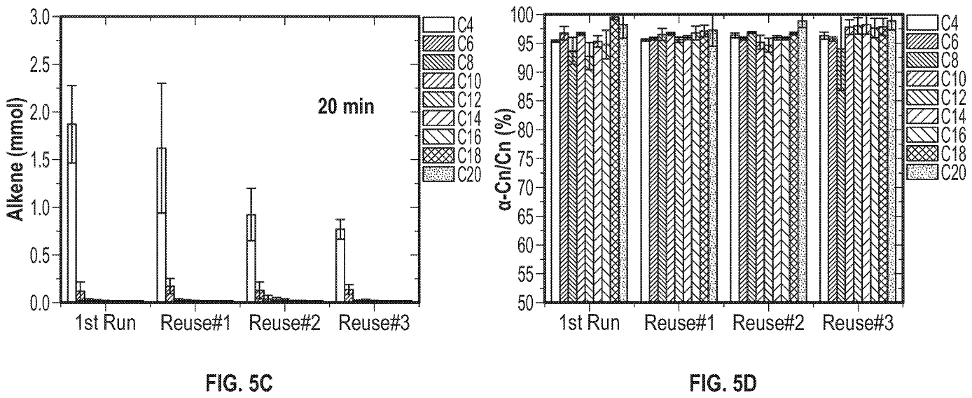

Ethylene oligomerization using heterogenous Fe-4 supported catalyst was examined in the context of a batch reactor with reduced reaction time, in comparison with Example 12. It was observed from the results of prior examples that an exotherm present during the reaction was most significant during the first 20 minutes of the reaction. Example 17 was conducted using a shortened run times of 20 minutes to examine whether the increased exotherm observed in this initial stage of the oligomerization would correlate to an increased productivity. Conditions of the oligomerization were otherwise identical to those of Example 12, also shown in FIG. 5A for direct comparison. As shown in FIG. 5C, the productivity for Example 17 (“20 min”, right) showed only an approximately 50% decrease during the first and second oligomerization runs compared to Example 12 (“1 hour”, left), despite the reaction time being reduced by a factor of three.

Surprisingly, it was also observed that the productivity of subsequent oligomerizations demonstrated a less severe decrease, suggesting that the catalyst activity in subsequent runs may be preserved by shortening reaction times of individual oligomerizations. Indeed, the productivity of the second and third “reuse” of the catalyst for Example 16 (*estimated 0.9 mmol C₄, 0.8 mmol C₄, respectively) exceeded that observed for Example 12 despite having shorter run times. Further, selectivity to lighter olefins was also improved by the shorter reaction time, with the Example 17 producing 98 mol % C₄ and C₆ oligomers as compared to only 89 mol % for Example 12.

### Example 18: Ethylene Oligomerization Processes in a Flow Reactor with Liquid Trap

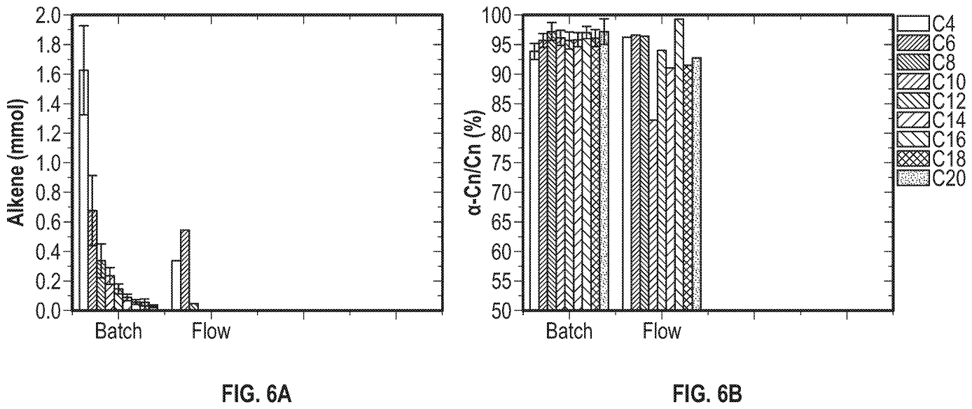

Ethylene oligomerization using heterogenous Fe-2 supported catalyst was examined in the context of a flow reactor. In this example, the reactor exhaust was partially opened to mimic the conditions of a flow reactor where unreacted ethylene is continually cycled through the reactor for the entirety of the reaction. A cooled liquid trap (−15 °C) was assembled during the reaction to collect the products. In this example, 580 mmol ethylene was flowed through the reactor over the course of an hour (a flow rate of approximately 233 SCCM). As shown in FIG. 6A, the oligomerization under flow condition resulted in less production of 1-butene as compared to the batch reactor setup of Example 11, which was mostly caused by the loss of 1-butene in the ethylene flow. The production of 1-hexene remained largely unaffected while the production of longer chain olefins was greatly reduced. C₄ and C₆ olefins represented 94% of the product mixture from the flow reactor setup, compared to only 71% for Example 11 (batch reactor).

### Examples 19-22: Ethylene Oligomerization Processes in a Flow Reactor with Liquid Trap, with Varied Ethylene Flow Rate and Reaction Time

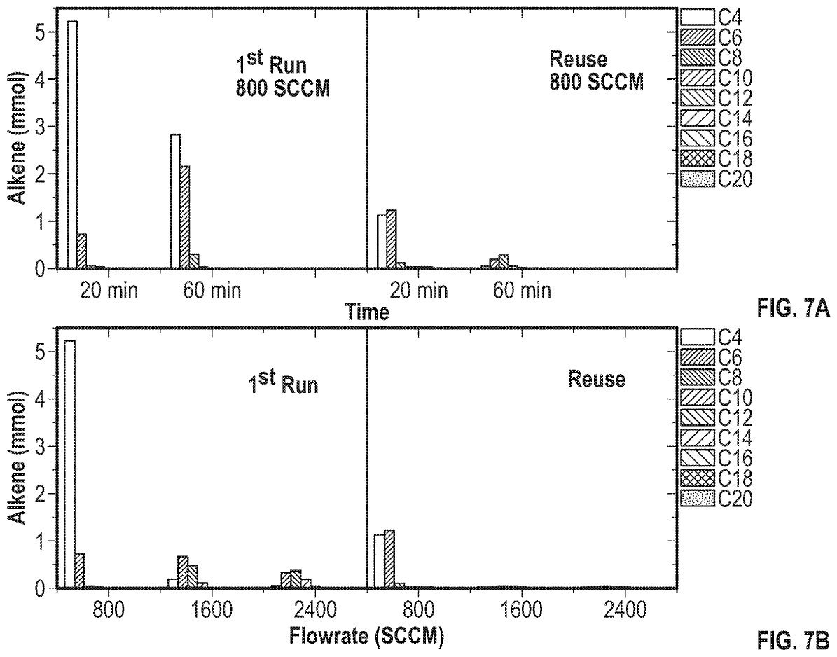

Additional experiments were conducted to observe the effect of ethylene flow rate on productivity using heterogenous Fe-4 supported catalyst. The reactions were performed using similar product collection methods to Example 18 using a cooled liquid trap, however, the reactor pressure was controlled by an electronic pressure controller and the flowrate was controlled by a high-pressure mass flow controller. Example 19 and Example 20 were tested at 200 psig of ethylene pressure with 800 SCCM flowrate, with 1 hour and 20 minutes reaction time respectively. As shown in FIG. 7A, under flow conditions the longer reaction time leads to significant loss of collected C₄ olefins in the cooled liquid trap. The amount of collected less volatile C₆ olefins is about 3-fold in a 60 min reaction time compared to 20 min. The reactivity greatly decreases in the reuse test with 60 min reaction time.

The flow rate was increased to 800 SCCM in Example 20, 1600 SCCM in Example 21, and 2400 SCCM in Example 22. As shown in FIG. 7B, a higher flow rate led to a significant loss of C₄ olefins. The amount of collected C₆ olefins remained similar for reactions using 800 and 1600 SCCM flowrate, but decreased when the flowrate was increased to 2400 SCCM, demonstrating a beneficial range for ethylene flow rate.

TABLE II
Summary of preparation and oligomer product characterization from
Examples 15-22. The catalyst was loaded to solid support via IWI.

| | | Al activator | Support | | Flowrate | Reaction | | α-C₄/C₄ | C₄/Cₙ | α-C₆/C₆ | C₆/Cₙ | C₄₋₆/Cₙ |
|---|---|---|---|---|---|---|---|---|---|---|---|---|
| Example | Catalyst | (equiv.) | (mass) | Reactor | (SCCM) | time | | (%) | (mol %) | (%) | (mol %) | (mol %) |
| 15 | Fe-2 | MMAO-3A | γ-Al₂O₃ | Batch | — | 1 | h | 92 | 62 | 95 | 18 | 80 |
| 1^{st} Run | | (500 eq.) | (2.5 g) | | | | | | | | | |
| 15 | Fe-2 | MMAO-3A | γ-Al₂O₃ | Batch | — | 1 | h | 92 | 56 | 94 | 18 | 74 |
| Reuse#1 | | (500 eq.) | (2.5 g) | | | | | | | | | |
| 16 | Fe-2 | MMAO-3A | γ-Al₂O₃ | Batch | — | 1 | h | 94 | 85 | 95 | 10 | 95 |
| 1^{st} Run | | (500 eq.) | (2.5 g) | | | | | | | | | |
| 16 | Fe-2 | MMAO-3A | γ-Al₂O₃ | Batch | — | 1 | h | 94 | 84 | 96 | 11 | 95 |
| Reuse#1 | | (500 eq.) | (2.5 g) | | | | | | | | | |
| 17 | Fe-4 | MMAO-3A | γ-Al₂O₃ | Batch | — | 20 | min | 95 | 93 | 97 | 5 | 98 |
| | | (500 eq.) | (2.5 g) | | | | | | | | | |
| 18 | Fe-2 | MMAO-3A | γ-Al₂O₃ | Flow | 233 | 1 | h | 96 | 36 | 97 | 58 | 94 |
| | | (500 eq.) | (2.5 g) | | | | | | | | | |
| 19 | Fe-4 | MMAO-3A | γ-Al₂O₃ | Flow | 800 | 1 | h | 97 | 54 | 96 | 41 | 94 |
| | | (500 eq.) | (2.5 g) | | | | | | | | | |
| 20 | Fe-4 | MMAO-3A | γ-Al₂O₃ | Flow | 800 | 20 | min | 94 | 87 | 93 | 12 | 90 |
| | | (500 eq.) | (2.5 g) | | | | | | | | | |
| 21 | Fe-4 | MMAO-3A | γ-Al₂O₃ | Flow | 1600 | 20 | mm | 99 | 13 | 96 | 46 | 59 |
| | | (500 eq.) | (2.5 g) | | | | | | | | | |
| 22 | Fe-4 | MMAO-3A | γ-Al₂O₃ | Flow | 2400 | 20 | min | 99 | 6 | 97 | 33 | 40 |

| | |
|---|---|
| (500 eq.) | (2.5 g) |

### Examples 23-29: Ethylene Oligomerization Processes in a Flow Reactor, Quantification Via Gas Phase

Flow reactions using heterogenous Fe-4 supported catalyst were performed with an alternative flow reactor design, which maintained the olefin products in the gas-phase without a trap to collect the products into liquid phase. An ethylene supply with a fixed ratio of internal standard was used for quantification of olefin products via GC analysis. Under the tested conditions using the modified flow reactor, the reactions were unexpectedly selective for C₄ and C₆ olefin products, with almost no longer chain products (<1%) detected.

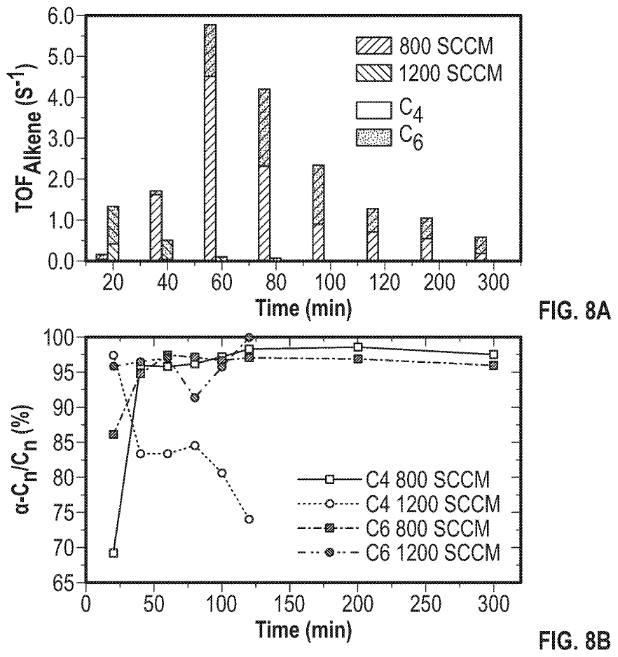

Examples 23-24 compared the effects of different flow rates (800 vs. 1200 SCCM, respectively). As shown in FIGS. 8A-8B, the higher flow rate deactivated the catalyst faster, while at the initial 20 min higher flow rate achieved better selectivity for terminal olefin.

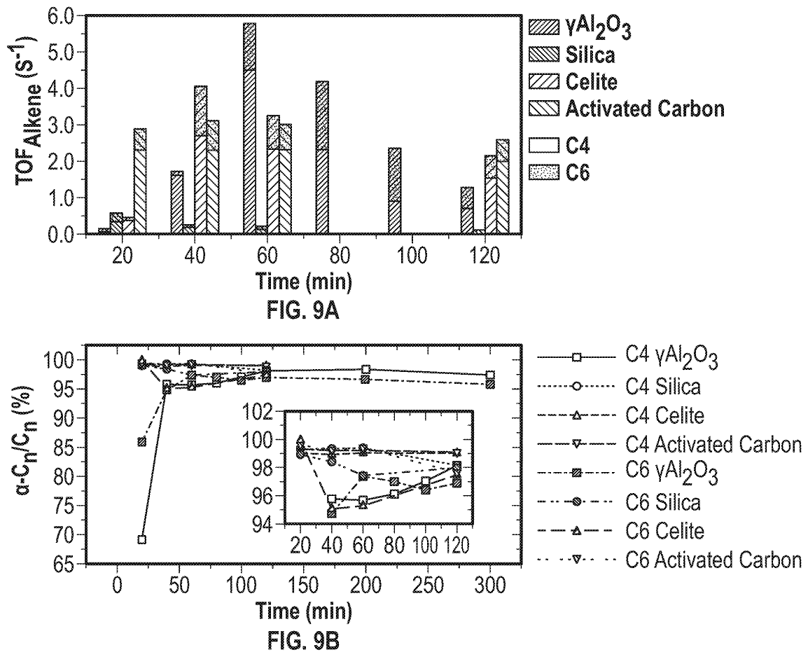

Examples 25-27 examined different solid support materials under the same conditions as Example 23. As shown in FIG. 9A and FIG. 9B, the identity of solid support materials affected both the reaction rate and the selectivity. Among the tested supports, activated carbon gave the best selectivity and longevity for the oligomerizations and maintained a satisfactory reaction rate. The activated carbon had a pore volume of 0.4-0.5 mL/g and a BET surface area in the 700-850 m²/g range.

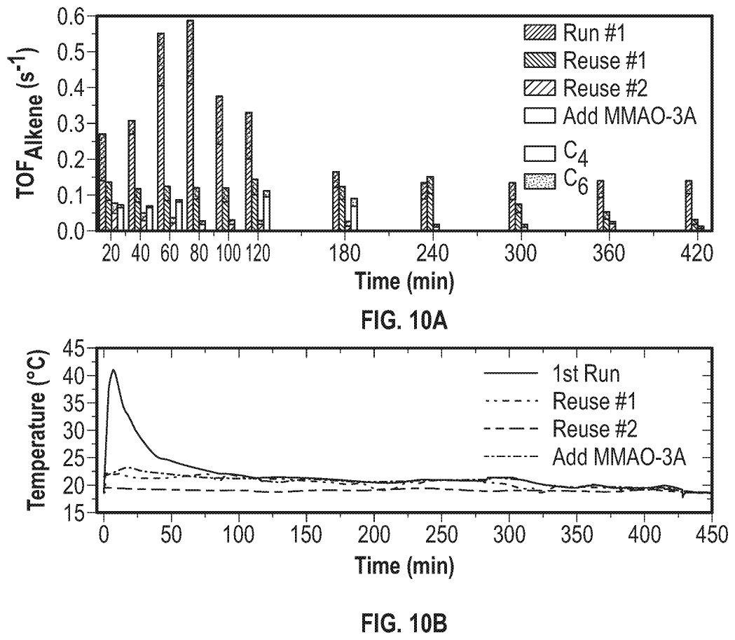

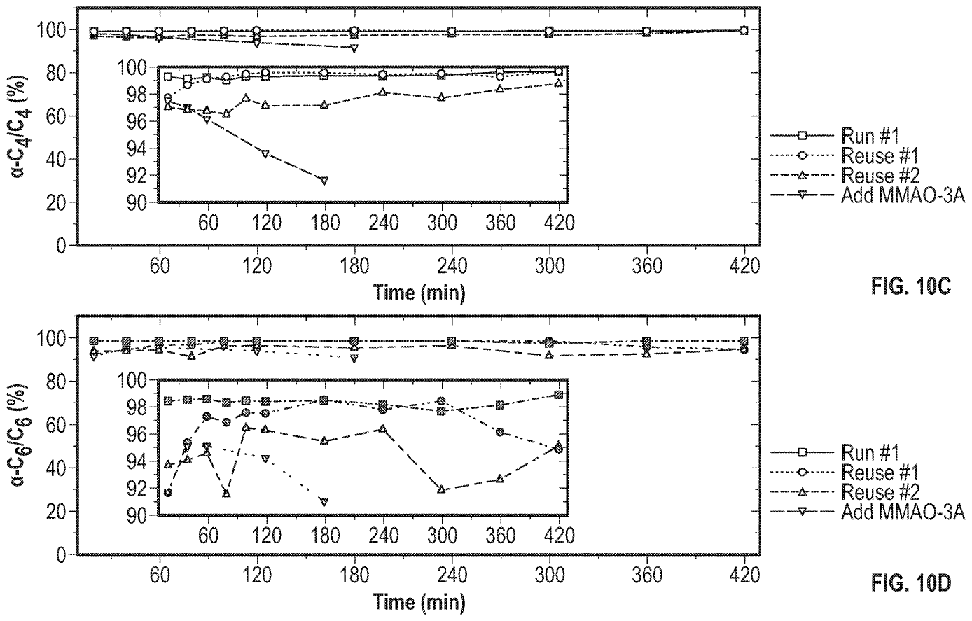

Additional experiments were conducted which further examined the longevity and reusability of heterogenous Fe-4 supported on activated carbon. Example 28 demonstrated longevity and reusability of the catalyst composition. As shown in FIG. 10A and FIG. 10B, the reaction showed best activity around 60-80 mins which, without being bound by theory, may be due to the increased temperature caused by the exothermic nature of the reaction. The reaction was tested continuously for seven hours, and a selectivity of 99% for terminal olefins was observed without any significant catalyst deactivation. After that, the catalyst chamber was purged using cylinder nitrogen and the active catalyst was stored under nitrogen prior to its reuse.

During the first reuse of the active catalyst, the observed activity was similar to the previous run for the first 4 hours and slowly decreased thereafter. The selectivity experienced a slight initial decrease but ramped up to 99% as the reaction proceeded. After the second reuse of the catalyst, the catalyst was reactivated using MMAO-3A under nitrogen atmosphere and dried via vacuum before use. An increase in reactivity was observed when using the reactivated catalyst compared to the previous run before reactivation, however, it was still lower than the first run. Selectivity for terminal olefin using reactivated catalyst slightly decreased in the second reuse from 99% to 92%.

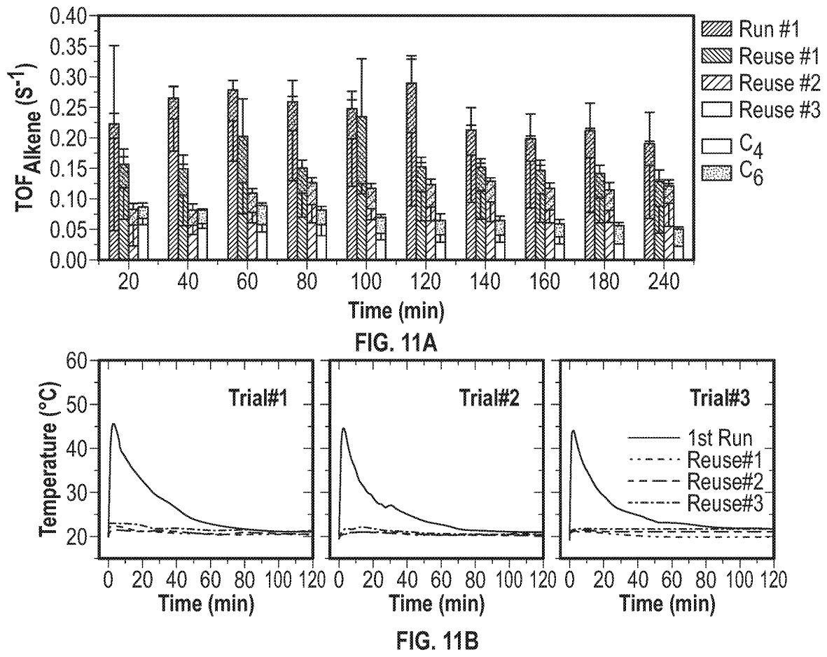

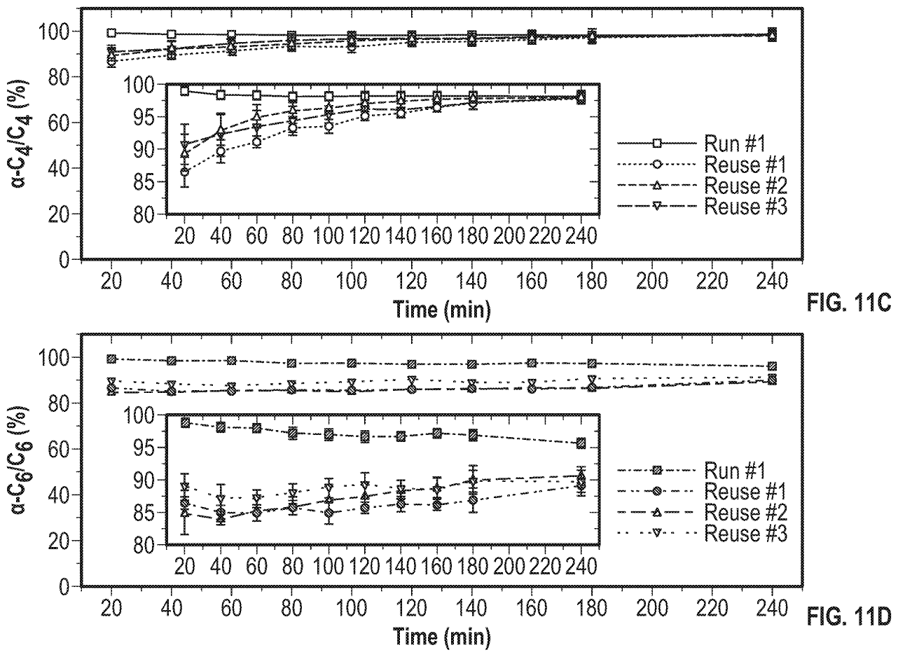

In Example 29, the activated heterogenous Fe-4 supported catalyst was made in large scale and used portion-wise based on the theoretical maximum Fe loading to match the earlier conditions (9.05 μmol for each reaction). As shown in FIGS. 11A-11D, the catalyst was examined during a continuous run of 4 hours, during which the activity and selectivity remained relatively constant. The catalyst chamber was then purged using cylinder nitrogen, and the active catalyst was stored under nitrogen before reuse. During the first reuse of the catalyst, the activity slightly decreased compared to the first run, unlike the continuous run of Example 28 during which the activity at 4 and 7 hours was nearly identical.

Without being bound by theory, it is believed that storage of the catalyst under nitrogen and/or the absence of ethylene slowly deactivates the catalyst. As for Example 28, the selectivity of Example 29 initially decreased before increasing to 99% as the reaction proceeded. Using the same procedure, when the active catalyst was stored and reused for a total of three times, the activity slowly decreased but the reaction still produced oligomerization products with similar high selectivity to terminal olefins.

TABLE III
Summary of preparation and oligomer product characterization from
Examples 23-29. The catalyst was loaded to solid support via IWI.

| | | Al activator | Support | | Pressure | Flowrate | Reaction | α-C₄/C₄ | C₄/Cₙ | α-C₆/C₆ | C₆/Cₙ |
|---|---|---|---|---|---|---|---|---|---|---|---|
| Example | Catalyst | (equiv.) | (mass) | Reactor | (psig) | (SCCM) | time | (%) | (mol %) | (%) | (mol %) |
| 23 | Fe-4 | MMAO-3A | γ-Al₂O₃ | Flow | 200 | 800 | 20 min | 69 | 35 | 86 | 65 |
| | | (500 eq.) | (2.5 g) | | | | | | | | |
| 23 | Fe-4 | MMAO-3A | γ-Al₂O₃ | Flow | 200 | 800 | 40 min | 96 | 95 | 95 | 5 |
| | | (500 eq.) | (2.5 g) | | | | | | | | |
| 24 | Fe-4 | MMAO-3A | γ-Al₂O₃ | Flow | 200 | 1200 | 20 min | 97 | 31 | 96 | 69 |
| | | (500 eq.) | (2.5 g) | | | | | | | | |
| 24 | Fe-4 | MMAO-3A | γ-Al₂O₃ | Flow | 200 | 1200 | 40 min | 83 | 11 | 96 | 89 |
| | | (500 eq.) | (2.5 g) | | | | | | | | |
| 25 | Fe-4 | MMAO-3A | Silica | Flow | 200 | 800 | 20 min | 99 | 57 | 99 | 43 |
| | | (500 eq.) | (1.0 g) | | | | | | | | |
| 25 | Fe-4 | MMAO-3A | Silica | Flow | 200 | 800 | 40 min | 99 | 73 | 98 | 27 |
| | | (500 eq.) | (1.0 g) | | | | | | | | |
| 26 | Fe-4 | MMAO-3A | Celite | Flow | 200 | 800 | 20 min | 99 | 84 | 95 | 16 |
| | | (500 eq.) | (1.0 g) | | | | | | | | |
| 26 | Fe-4 | MMAO-3A | Celite | Flow | 200 | 800 | 40 min | 99 | 67 | 95 | 33 |
| | | (500 eq.) | (1.0 g) | | | | | | | | |
| 27 | Fe-4 | MMAO-3A | Activated | Flow | 200 | 800 | 20 min | 99 | 80 | 99 | 20 |
| | | (500 eq.) | Carbon (3.5 g) | | | | | | | | |
| 27 | Fe-4 | MMAO-3A | Activated | Flow | 200 | 800 | 40 min | 99 | 74 | 99 | 26 |
| | | (500 eq.) | Carbon (3.5 g) | | | | | | | | |
| 28 | Fe-4 | MMAO-3A | Activated | Flow | start | 100 | 40 min | 99 | 87 | 99 | 13 |
| | | (500 eq.) | Carbon | | from 150 | | | | | | |
| 28 | Fe-4 | MMAO-3A | Activated | Flow | start | 100 | 420 min | 99 | 73 | 98 | 27 |
| | | (500 eq.) | Carbon | | from 150 | | | | | | |
| 29 | Fe-4 | MMAO-3A | Activated | Flow | 200 | 100 | 40 min | 99 | 88 | 99 | 12 |
| | | (500 eq.) | Carbon | | | | | | | | |

### Examples 30-41: Cobalt Catalyst Compositions for Ethylene Oligomerization

Cobalt complexes having structures consistent with Formula (III) were prepared to exemplify the characteristics of the improved supported catalyst compositions and their use within ethylene oligomerization processes. Various catalyst compositions were evaluated for their effectiveness in oligomerization processes to selectively produce light α-oligomers.

For each of Examples 30-35, a catalyst composition comprising an NNN cobalt complex (Co-1) was prepared by first dissolving Co-1 (5.5 mg, 9.05 μmol) in 2 mL toluene. MMAO (500 eq.) was then added to the solution to activate Co-1 catalyst. For Examples 36-41, a catalyst composition was prepared comprising an alternate NNN cobalt complex (Co-2) instead of Co-1. For examples employing a heterogenous catalyst composition (Examples 33-35 and 39-41), the catalyst composition was loaded onto the appropriate solid support by incipient wet impregnation as described above to yield a heterogenous oligomerization catalyst composition.

Alternatively, Examples 30-32 and 36-38 employed a homogenous catalyst and therefore were not loaded onto a solid support. For Examples 32 and 38 (indicated as “no support” in Table IV), the Co/MMAO mixture solution was dried inside the Fisher porter reactor until forming a solid and then directly used for the oligomerization reactions. For Examples 30-31 and 36-37 (indicated as “in toluene” in Table IV), the Co/MMAO mixture was not dried, and the Co/MMAO mixture solution was directly used for the oligomerization reaction.

In each of Examples 30-41, the appropriate catalyst composition was loaded into a batch reactor for ethylene oligomerization under the same conditions described above for Examples 2-8. The ethylene pressure was the same for each reaction using a Parr high pressure gas burette. Preparation methods and reaction conditions are summarized for each of Examples 30-41 in Table IV below. Products were analyzed by gas chromatography, using an internal standard, and the results are also shown in Table IV below.

Surprisingly, each of Examples 30-41 demonstrated a high selectivity to the production of C₄ and C₆ oligomers. This result occurred almost irrespective of whether the catalyst composition was heterogenous or homogenous in nature. Only a slight reduction in the amount of C₄ and C₆ oligomers produced by the heterogenous catalyst compositions of inventive Examples 33-35 and 39-41 was observed as compared to homogenous catalyst compositions. Comparative Examples 30-32 (Co-1) and 36-38 (Co-2) resulted in a C₄₋₆/Cₙ of at least 97.9% and in excess of 99%. Example 33 produced the lowest relative amount of C₄ and C₆ oligomers, but the oligomer product mixture contained 92.8% C₄ and C₆ oligomers. Example 33 using Co-1 supported on γ-Al₂O₃ gave only a slightly lower amount of C₄ and C₆ oligomers (97.8%) than examples using homogenous catalyst compositions. Examples 40-41 (Co-2), supported on 4A sieves and activated carbon respectively, each resulted in excess of 99% C₄ and C₆ oligomers.

Further, the amount of α-olefin within the C₄ and C₆ olefin products was also high for several inventive examples. Example 35 for instance, which used a Co-1 catalyst complex supported on activated carbon, yielded 94% α-C₄/C₄ and 84% α-C₆/C₆. These values are comparable to the homogenous catalyst composition of Comparative Example 31 under identical conditions, which yielded 99% α-C₄/C₄ and 96% α-C₆/C₆. Example 41, which used a Co-2 catalyst complex supported on activated carbon, yielded 90% α-C₄/C₄ and 91% α-C₆/C₆ also demonstrated a relatively high amount of the respective α-olefin products.

Thus, the heterogenous catalyst compositions of Examples 33-35 (Co-1) and Examples 39-41 (Co-2) both demonstrated similar productivity and selectivity to α-olefin products relative to the Comparative Examples 30-32 (Co-1) and Comparative Examples 36-38 (Co-2) where no support was included.

### Examples 42-43: Chromium Catalyst Compositions for Ethylene Oligomerization

Chromium complexes Cr-1 and Cr-2 each had structures consistent with Formula (II) and are shown below. Supported catalyst compositions comprising the chromium complexes were prepared to exemplify the characteristics of the improved supported catalyst compositions and their use within ethylene oligomerization processes. The catalyst compositions were evaluated for their effectiveness in oligomerization processes to selectively produce light α-oligomers.

Catalyst compositions comprising a PN-Cr complex was prepared by first dissolving the Cr complex (6 mg, 10.9 μmol) in 2 mL toluene. MMAO (500 eq.) was then added to the solution to activate Cr-1 catalyst. For Examples 42-43, the catalyst composition was loaded onto the appropriate solid support by incipient wet impregnation as described above to yield a heterogenous oligomerization catalyst composition. Each of the catalysts were packed into a flow reactor generally as described above for Examples 19-22, however, at a significantly reduced flow rate (Example 42 flow rate of approximately 107 SCCM; Example 43 flow rate of approximately 200 SCCM). The resulting products were evaluated according to the amount and selectivity of 1-butene, and 1-hexene produced.

As shown in Table IV, the PN-Cr catalyst composition of Example 42 resulted in 1-butene and 1-hexene with 70% and 82% selectivity, with respect to the amount of butene and hexene produced. Example 43 also resulted in a product mixture favoring the α-olefin products, with 58% of the butene produced being 1-butene, and 75% of the hexene produced being 1-hexene. Despite the reduced flow rates relative to prior examples, the total yield of butene and hexene in the product mixture for Examples 42 and 43 was 58% and 38% respectively.

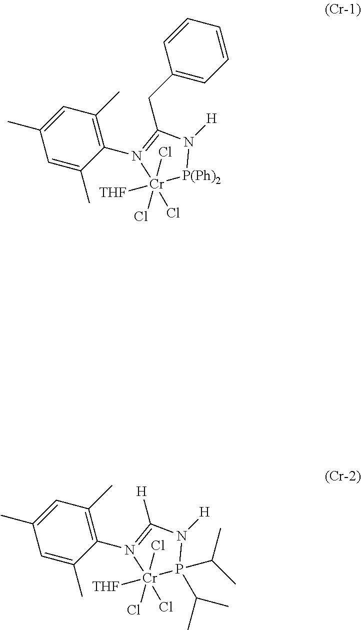

### Examples 44-45: Carbon Supported Catalyst Compositions for Ethylene Oligomerization

For Example 44, Fe-4 (31.5 mg) and 500 equivalents MMAO were supported on activated carbon (20 g) using the IWI method described above. The catalyst was packed into a flow reactor generally as described above and a flow rate of 100 SCCM and ethylene pressure of 100 psig. Table V summarizes the results for Example 44 on the first use of the catalyst and two reuse cycles.

For Example 45, Fe-4 (31.5 mg) and 500 equivalents MMAO were supported on Vulcan carbon black (20 g) using the IWI method described above. The catalyst was packed into a flow reactor generally as described above and a flow rate of 100 SCCM and ethylene pressure of 100 psig. Table VI summarizes the results for Example 45 on the first use of the catalyst and two reuse cycles.

TABLE IV
Summary of preparation and oligomer product characterization from Examples 30-43.

| | | Al activator | Support | | Reaction | | Catalyst | α-C₄/C₄ | C₄/Cₙ | α-C₆/C₆ | C₆/Cₙ | C₄₋₆/Cₙ |
|---|---|---|---|---|---|---|---|---|---|---|---|---|
| Example | Catalyst | (500 equiv.) | (mass) | Reactor | time | | loading | (%) | (mol %) | (%) | (mol %) | (mol %) |
| 30 | Co-1 | MMAO-3A | in toluene | Batch | 4 | h | N/A | >99 | 97.6 | 91 | 2.4 | >99 |
| 31 | Co-1 | MMAO-3A | in toluene | Batch | 24 | h | N/A | 99 | 93.5 | 96 | 5.1 | 98.6 |
| 32 | Co-1 | MMAO-3A | no support | Batch | 24 | h | N/A | 86 | 92.8 | 70 | 5.7 | 98.5 |
| 33 | Co-1 | MMAO-3A | γ-Al₂O₃ | Batch | 24 | h | IWI | 77 | 92.5 | 75 | 5.3 | 97.8 |
| | | | (2.5 g) | | | | | | | | | |
| 34 | Co-1 | MMAO-3A | 4A sieves | Batch | 24 | h | IWI | 15 | 93.5 | 12.5 | 2.9 | 96.4 |
| 35 | Co-1 | MMAO-3A | Activated | Batch | 24 | h | IWI | 94 | 85.8 | 89 | 7.0 | 92.8 |
| | | | Carbon | | | | | | | | | |
| 36 | Co-2 | MMAO-3A | in toluene | Batch | 4 | h | N/A | 99 | >99 | — | — | >99 |
| 37 | Co-2 | MMAO-3A | in toluene | Batch | 24 | h | N/A | >99 | 93.2 | 94 | 6.8 | >99 |
| 38 | Co-2 | MMAO-3A | no support | Batch | 24 | h | N/A | 87 | 94.8 | 81 | 3.1 | 97.9 |
| 39 | Co-2 | MMAO-3A | γ-Al₂O₃ | Batch | 24 | h | IWI | 50 | 86.7 | 47 | 10.4 | 97.1 |
| | | | (2.5 g) | | | | | | | | | |
| 40 | Co-2 | MMAO-3A | 4A sieves | Batch | 24 | h | IWI | 50 | 96.5 | 39 | 3.5 | >99 |
| | | | (2.5 g) | | | | | | | | | |
| 41 | Co-2 | MMAO-3A | Activated | Batch | 24 | h | IWI | 90 | 94.8 | 91.0 | 5.2 | >99 |
| | | | Carbon | | | | | | | | | |
| 42 | Cr-1 | MMAO-3A | γ-Al₂O₃ | Flow | 1 | h | IWI | 70 | 25 | 82 | 33 | 58 |
| | | | (2.5 g) | | | | | | | | | |
| 43 | Cr-2 | MMAO-3A | γ-Al₂O₃ | Flow | 20 | m | IWI | 58 | 13 | 75 | 25 | 38 |

(2.5 g)

TABLE V
Summary of Example 44.

| | | | | |
|---|---|---|---|---|
| Time | C₄ TOF | C₆ TOF | α-C₄/C₄ | α-C₆/C₆ |
| (min) | (/s) | (/s) | (%) | (%) |

First Use

| | | | | |
|---|---|---|---|---|
| 20 | 0.232 | 0.040 | 99.25 | 99.46 |
| 40 | 0.152 | 0.044 | 99.26 | 99.29 |
| 60 | 0.120 | 0.046 | 99.28 | 99.23 |
| 80 | 0.102 | 0.047 | 99.34 | 99.21 |
| 100 | 0.088 | 0.047 | 99.36 | 99.15 |
| 120 | 0.079 | 0.044 | 99.39 | 99.16 |

Reuse#1

| | | | | |
|---|---|---|---|---|
| 20 | 0.351 | 0.058 | 99.46 | 94.84 |
| 40 | 0.218 | 0.066 | 99.29 | 95.61 |
| 60 | 0.156 | 0.062 | 99.23 | 96.29 |
| 80 | 0.121 | 0.057 | 99.21 | 96.37 |
| 100 | 0.098 | 0.053 | 99.15 | 95.86 |
| 120 | 0.083 | 0.049 | 99.16 | 96.38 |

Reuse#2

| | | | | |
|---|---|---|---|---|
| 20 | 0.076 | 0.024 | 99.46 | 92.61 |
| 40 | 0.056 | 0.023 | 99.34 | 95.29 |
| 60 | 0.044 | 0.022 | 99.20 | 95.93 |
| 80 | 0.035 | 0.019 | 98.98 | 96.16 |
| 100 | 0.030 | 0.017 | 98.21 | 96.63 |
| 120 | 0.026 | 0.016 | 98.82 | 96.63 |

TABLE VI
Summary of Example 45.

| | | | | |
|---|---|---|---|---|
| Time | C₄ TOF | C₆ TOF | α-C₄/C₄ | α-C₆/C₆ |
| (min) | (/s) | (/s) | (%) | (%) |

First Use

| | | | | |
|---|---|---|---|---|
| 20 | 0.112 | 0.054 | 98.75 | 94.22 |
| 40 | 0.041 | 0.029 | 98.97 | 94.94 |
| 60 | 0.029 | 0.022 | 98.93 | 98.93 |
| 80 | 0.025 | 0.018 | 98.79 | 99.00 |
| 100 | 0.021 | 0.015 | 98.52 | 99.02 |
| 120 | 0.018 | 0.012 | 98.47 | 98.77 |

Reuse#1

| | | | | |
|---|---|---|---|---|
| 20 | 0.109 | 0.019 | 98.21 | 94.69 |
| 40 | 0.041 | 0.019 | 97.89 | 94.79 |
| 60 | 0.022 | 0.017 | 98.06 | 93.95 |

Reuse#2

| | | | | |
|---|---|---|---|---|
| 20 | 0.014 | 0.005 | 98.19 | 89.86 |
| 40 | 0.007 | 0.006 | 97.16 | 92.53 |
| 60 | 0.005 | 0.004 | 96.52 | 92.48 |
| 80 | 0.003 | 0.003 | 95.75 | 87.89 |
| 100 | 0.003 | 0.003 | 95.92 | 84.54 |
| 120 | 0.003 | 0.002 | 94.76 | 82.65 |

What is claimed is:

1. An oligomerization process comprising:
(I) contacting ethylene, a catalyst composition, and optionally hydrogen, in an oligomerization reactor;
(II) forming an oligomer product in the oligomerization reactor, the oligomer product comprising C₄ to C₂₀ ethylene oligomers; and
(III) discharging an effluent stream from the oligomerization reactor, the effluent stream comprising unreacted ethylene and the oligomer product;
wherein the catalyst composition comprises:
an activator;
a solid support; and
a heteroatomic ligand transition metal complex having Formula (II): 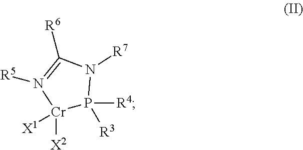
wherein:
X¹ and X² independently are a monoanionic ligand; and
R³, R⁴, R⁵, R⁶, and R⁷ independently are a C₁ to C₂₀ organyl group.

2. The oligomerization process of claim 1, wherein:
R⁶ is NR^{6a}R^{6b}, and
R^{6a} and R^{6b} independently are a C₁ to C₂₀ organyl group, each optionally forming a linking group with R⁵, R⁷, or each other.

3. The oligomerization process of claim 1, wherein:
R³ and R⁴ independently are a substituted or unsubstituted C₆-C₁₂ aryl group; and
X¹ and X² are Cl.

4. The oligomerization process of claim 1, wherein the activator comprises an aluminoxane selected from methylaluminoxane (MAO), ethylaluminoxane, modified methylaluminoxane (MMAO), isobutyl-modified methyl aluminoxane, n-propylaluminoxane, iso-propyl-aluminoxane, n-butylaluminoxane, sec-butylaluminoxane, iso-butylaluminoxane, t-butylaluminoxane, 1-pentylaluminoxane, 2-pentylaluminoxane, 3-pentylaluminoxane, iso-pentylaluminoxane, neopentylaluminoxane, and any combination thereof.

5. The oligomerization process of claim 1, wherein the solid support comprises a solid oxide, a molecular sieve, a zeolite, a diatomaceous earth, a carbon material, or any combination thereof.

6. The oligomerization process of claim 1, wherein the solid support comprises a carbon black, an activated carbon, a vulcanized carbon, a carbon nanomaterial, or any combination thereof.

7. The oligomerization process of claim 1, wherein the solid support is characterized by:
a BET surface area in a range from 2 to 1500 m²/g;
a pore volume in a range from 0.2 to 5 mL/g; and
a d50 average particle size in a range from 5 to 500 μm.

8. The oligomerization process of claim 1, wherein:
an amount of transition metal on the solid support is in a range from 0.001 to 10 wt. %; and/or
a molar ratio of aluminum to transition metal in the catalyst composition is in a range from 10:1 to 5,000:1.

9. The oligomerization process of claim 1, further comprising, prior to step (I):
(a) contacting the heteroatomic ligand transition metal complex and the activator to form an activated catalyst;
(b) contacting the activated catalyst and the solid support to form a supported catalyst; and
(c) drying the supported catalyst to form the catalyst composition.

10. The oligomerization process of claim 1, wherein the oligomerization reactor comprises a fixed bed reactor, and the ethylene contacts a fixed bed of the catalyst composition in step (I).

11. The oligomerization process of claim 1, wherein the oligomerization reactor comprises a continuous stirred tank reactor or a loop slurry reactor.

12. The oligomerization process of claim 1, wherein:
the ethylene is a liquid in step (I); or
the ethylene is a gas in step (I).

13. The oligomerization process of claim 1, wherein:
the unreacted ethylene is a liquid in step (III);
the oligomer product is a liquid in step (III);
the oligomer product comprises C₆ to C₈ olefins; or
any combination thereof.

14. The oligomerization process of claim 1, further comprising:
a step of removing the unreacted ethylene from the effluent stream by evaporating or flashing under reduced pressure;
a step of separating the catalyst composition and the oligomer product, or a step of separating the catalyst composition and the effluent stream, by draining, decanting, pressing, centrifuging, filtering, sedimenting, or any combination thereof, or
both.

15. The oligomerization process of claim 1, wherein:
the oligomerization process has a single pass ethylene conversion of from 5 to 95 mol %;
the oligomerization process has an ethylene conversion of from 20 to 95 mol %;
the oligomer product comprises at least 65 mol % of α-olefin oligomers, based on a total amount of oligomers in the oligomer product; or
any combination thereof.

16. The oligomerization process of claim 1, wherein a productivity of the catalyst composition (in kg (C₄ to C₂₀ olefin)/g transition metal, in kg (C₄ to C₂₀ olefin)/g transition metal/hr, or in kg (C₄ to C₂₀ olefin)/g Al) is within 25% of that of an otherwise identical process using an otherwise identical homogenous catalyst system.

17. The oligomerization process of claim 1, further comprising a second oligomerization process reusing the catalyst composition, the second oligomerization process comprising:
(IV) optionally, regenerating the catalyst composition within the oligomerization reactor;
(V) contacting ethylene and optionally hydrogen in the oligomerization reactor with the catalyst composition;
(VI) forming a second oligomer product in the oligomerization reactor, the second oligomer product comprising C₄ to C₂₀ ethylene oligomers; and
(VII) discharging a second effluent stream from the oligomerization reactor, the second effluent stream comprising second unreacted ethylene and the second oligomer product.

18. The oligomerization process of claim 17, wherein:
the ethylene in step (V) is the unreacted ethylene recovered from step (III);
an activity, productivity, and/or selectivity of the catalyst composition in step (VI) of the second oligomerization process is in a range from 20% to 200% of that of the respective activity, productivity, and/or selectivity of the catalyst composition in step (II) of the oligomerization process;
a single pass ethylene conversion in steps (V)-(VII) is within 10%, within 25%, or within 40%, of the single pass ethylene conversion of steps (I)-(III), under the same oligomerization reaction conditions; or
any combination thereof.

19. An oligomerization process comprising:
(I) contacting ethylene, a catalyst composition, and optionally hydrogen, in an oligomerization reactor;
(II) forming an oligomer product in the oligomerization reactor, the oligomer product comprising C₄ to C₂₀ ethylene oligomers; and
(III) discharging an effluent stream from the oligomerization reactor, the effluent stream comprising unreacted ethylene and the oligomer product;
wherein the catalyst composition comprises:
an activator;
a solid support; and
a heteroatomic ligand transition metal complex having Formula (I): 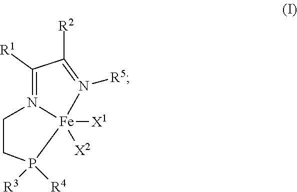
wherein:
X¹ and X² independently are a monoanionic ligand;
R¹ and R² independently are H or a substituted or unsubstituted C₁ to C₁₈ hydrocarbyl group; and
R³ and R⁴ independently are a substituted or unsubstituted C₁ to C₂₀ organyl group; or
a heteroatomic ligand transition metal complex having Formula (III): 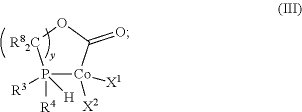
wherein:
X¹ and X² independently are a monoanionic ligand;
R³ and R⁴ independently are a substituted or unsubstituted C₁ to C₂₀ organyl group;
y is 0, 1, or 2; and
each R⁸, when present, independently is H or a substituted or unsubstituted C₁ to C₁₈ hydrocarbyl group; or
a heteroatomic ligand transition metal complex selected from:

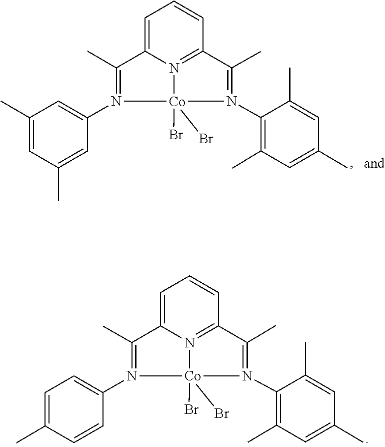

20. A process for preparing a catalyst composition, the process comprising:
(a) contacting a heteroatomic ligand transition metal complex and an activator to form an activated catalyst;
(b) contacting the activated catalyst and a solid support to form a supported catalyst; and
(c) drying the supported catalyst to form the catalyst composition.

21. A catalyst composition comprising:
an activator;
a solid support; and
a heteroatomic ligand transition metal complex having Formula (II): 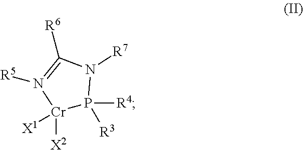
wherein:
X¹ and X² independently are a monoanionic ligand; and
R³, R⁴, R⁵, R⁶, and R⁷ independently are a C₁ to C₂₀ organyl group; or
a heteroatomic ligand transition metal complex having Formula (I): 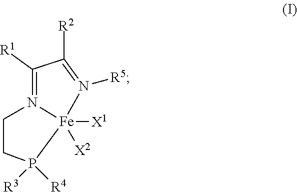
wherein:
X¹ and X² independently are a monoanionic ligand;
R¹ and R² independently are H or a substituted or unsubstituted C₁ to C₁₈ hydrocarbyl group; and
R³ and R⁴ independently are a substituted or unsubstituted C₁ to C₂₀ organyl group; or
a heteroatomic ligand transition metal complex having Formula (III): 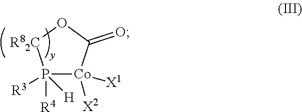
wherein:
X¹ and X² independently are a monoanionic ligand;
R³ and R⁴ independently are a substituted or unsubstituted C₁ to C₂₀ organyl group;
y is 0, 1, or 2; and
each R⁸, when present, independently is H or a substituted or unsubstituted C₁ to C₁₈ hydrocarbyl group; or
a heteroatomic ligand transition metal complex selected from: [chemical structure]
